(19) United States
(12) Patent Application Publication
(10) Pub. No.: US 2015/0190151 A1
(43) Pub. Date: Jul. 9, 2015

(54) SURGICAL GUIDE WITH INTRAOPERATIVE DEPTH FEEDBACK

(71) Applicant: Smith & Nephew, Inc., Memphis, TN (US)

(72) Inventors: Sachin P. Budhabhatti, Collierville, TN (US); Nathaniel M. Quinn, Arlington, TN (US); David C. Kelman, Collierville, TN (US); Alisha W. Bergin, Southaven, MS (US)

(21) Appl. No.: 14/390,705

(22) PCT Filed: Apr. 3, 2013
(86) PCT No.: PCT/US2013/035119

§ 371 Date: Oct. 3, 2014

Related U.S. Application Data
(60) Provisional application No. 61/620,227, filed on Apr. 4, 2012.
(60) Provisional application No. 61/623,995, filed on Apr. 13, 2012.

(51) Int. Cl. A61B 17/17

(52) U.S. Cl. CPC A61B 17/17; A61B 17/1746; A61B 2017/1602

(57) ABSTRACT

Disclosed herein are systems, devices, and methods for guiding surgical instruments to prepare a bone surface for joint reconstruction or repair. Examples include a surgical guide having a base and at least one linearly repositionable leg that provides depth tactile feed-back for an orthopedic preparation device, such as a reamer or impactor. Other example devices and systems include a surgical guide that operates with a positive depth stop for an orthopedic reaming or impacting device. The guide preferably includes a surface that forms a complementary fit with a specific portion of patient anatomy.

### CROSS-REFERENCE TO RELATED APPLICATIONS

This application claims the benefit of U.S. Provisional Application No. 61/620,227, filed Apr. 4, 2012, and U.S. Provisional Application No. 61/623,995, filed Apr. 13, 2012, which are hereby incorporated by reference herein in their entireties.

### BACKGROUND

Joints undergo degenerative changes for a variety of reasons. When joint degeneration becomes advanced or irreversible, it may become necessary to replace the natural joint with a prosthetic joint. Artificial implants, including hip joints, shoulder joints, and knee joints are widely used in orthopedic surgery. Specifically, hip joint prostheses are common. The human hip joint acts mechanically as a ball and socket joint, where the ball-shaped head of the femur is positioned within the socket-shaped acetabulum of the pelvis. Various degenerative diseases and injuries may require replacement of all or a portion of a hip or other joints using prosthetic implants constructed of metals, ceramics, plastics, or other synthetic materials.

Joint preparation for reconstruction and placement of a prosthetic implant typically requires re-shaping the joint by reaming, drilling, cutting, or other surgical procedures. For example, in a typical hip replacement procedure, the acetabulum is reshaped with a reamer. The reaming process removes sclerotic, arthritic bone to form a smooth, concave surface for tightly fitting a hemispherical prosthetic acetabular shell. Frequently, the head of the femur is also replaced with a prosthetic implant.

Proper bone shaping of the joint at the proper depth, angle, and diameter can be important for successful joint reconstruction and fixation of prosthetic implants. The re-shaped acetabulum surface determines the placement of the cup implant, which must be placed at an appropriate angle. Improper reaming of the acetabulum by excessive reaming or reaming in an errant angle can cause poor outcomes, including dislocation, pain, inflammation, leg length discrepancies, inflammation, nerve damage, implant failure, and revision surgeries.

Since each patient has a unique anatomy, the shaping process poses a significant challenge for surgeons, particularly in cases of substantial bone degeneration and defects. Standard freehand reaming and placement of an acetabular cup can be inconsistent. Surgical guides for the cutting, reaming, and drilling steps can help surgeons properly and accurately prepare the joint. Surgical guides that are adapted to individual patient anatomy can be particularly valuable for effective joint reconstruction.

### SUMMARY

Disclosed herein are systems, devices, and methods for guiding surgical instruments to prepare a bone surface for joint reconstruction or repair. The systems, devices, and methods disclosed herein incorporates features designed to limit movement of the orthopedic preparation device and thereby allow proper bone shaping of the joint at the proper depth, angle, and diameter to enable successful joint reconstruction and fixation of prosthetic implants. Some examples of the systems and devices disclosed include a surgical guide having a base and at least one linearly repositionable leg that provides tactile depth feedback for an orthopedic preparation device, such as a reamer. Linearly repositioning the leg allows the surgeon or technician to raise and lower the device for surgical application. A limiting depth stop may be included to limit the operational range within which the device can penetrate the patient. Other examples of the systems and devices disclosed include a base, guide hub, and positive depth stop. The guide hub is coupled to the base and has an aperture for guiding an orthopedic cutting or impacting device. The positive depth stop is structured to mate with the guide hub to limit movement of the orthopedic preparation device relative to the guide hub. Variations and combinations of these example surgical guide devices are also provided. Further disclosed herein are methods of making and using orthopedic surgical guides.

In certain embodiments, the structure of the guide is planned pre-operatively to form a patient-matched design that appropriately accounts for individual patient anatomy. The guide preferably includes a surface that forms a complementary fit with a specific portion of patient anatomy. The surface may be based on topographical data, such as MRI, X-ray, computed tomography (CT), or ultrasound images, of the patient's anatomy. For example, a reamer may be guided in a pre-planned insertion angle and depth relative to the surfaces of the joint, such as an acetabulum.

In certain embodiments, systems and devices for guiding a surgical preparation device, such as a reamer or impactor, include an orthopedic surgical guide, comprising at least one leg, a base, and a housing with an aperture slot, crevice, or other support feature that receives and aligns the device with respect to the acetabulum or other surgical site. An example of the housing includes a hub. In certain implementations, the leg has a longitudinal axis, a proximal end, a distal end, and a first leg component coupled to a second leg component, wherein the first leg component is linearly repositionable relative to the second leg component along the longitudinal axis. The second leg component may slide within the first leg component. The housing is coupled to the proximal end of the leg and its aperture receives the orthopedic cutting or impacting device. The leg may be releasably coupled to the hub. The leg may be repositionable relative to the hub. The base is disposed at the distal end of the leg. In certain embodiments, the leg has a surface structured to mate specifically with a portion of a patient's anatomy. Alternative embodiments include a plurality of legs.

Systems, devices, and methods provide linearly repositionable legs for a surgical guide. In certain embodiments, the guide has first and second leg components that are coupled with a spring, such as a compression spring composed of metal. In certain embodiments the spring is an elastomer. In alternative embodiments, the spring is a gas. The resistive force from compression of the spring provides tactile feedback for the movement of the orthopedic preparation device.

Further disclosed herein are systems, devices, and methods to provide a positive depth stop for a reamer to prevent excessive reaming. Examples include a guide, having at least one leg and a hub. Preferably, the leg has first and second leg components that are linearly repositionable within a predefined range of motion. In certain embodiments, the movement is limited by the compression of a spring. Alternatively, the first and second leg components may be coupled by means of a slot and ledge protrusion, where the movement is restricted by the length of the slot. The depth stop may be repositionable relative to the leg. For example, a depth stop may be provided with a removable, repositionable stop pin. The depth stop may also be an adjustable screw.

Further disclosed herein are systems and devices for guiding surgical instruments to prepare a bone surface for joint reconstruction. Examples include a leg, guide hub, and base. The base preferably forms a complementary fit with a portion of the patient's anatomy. In certain embodiments, the base is formed from topography data indicative of the portion of a patient's anatomy. The base may also include an opening for receiving a fastener, such as a bone pin. A drill guide may be used for accurately placing the surgical guide and base relative to the patient's anatomy.

Methods are also included for using an orthopedic surgical guide that provides tactile feedback and a depth stop to limit or otherwise direct the use of a surgical preparation device. Example implementations are provided by a surgical system having a base and a repositionable leg. The leg has a first leg component coupled and linearly repositionable relative to a second leg component. The leg may be coupled to a hub and to the base. The hub has an aperture that receives an orthopedic cutting or impacting device. The base has a surface structured to mate specifically with a portion of a patient's anatomy. Steps may include placing the base on the specific portion of the patient's anatomy, placing the orthopedic cutting or impacting device within the aperture of the guide hub, coupling an orthopedic cutting or impacting device within the aperture of the hub, sliding the first leg component along the longitudinal axis by applying a force on the orthopedic cutting or impacting device, and contacting the orthopedic cutting or impacting device with bone or tissue. The method may also include positioning a leg relative to the hub to adjust the angle between the leg and the hub. The method may also include sliding the first leg component relative to the second leg component until the limit of movement or depth stop is reached.

Disclosed herein are methods of making an orthopedic surgical guide. In certain example methods, the steps include collecting topography data of a portion of a patient's anatomy, creating a model of the patient's anatomy from the data, forming at least one leg, forming a guide hub with an aperture for guiding an orthopedic surgical preparation device, forming a base, and coupling at least one leg to the guide hub and base. In certain embodiments, the leg is provided with a longitudinal axis, a first leg component, and a second leg component, and the method includes coupling the first leg component with the second leg component along the longitudinal axis such that the first leg component is linearly repositionable relative to the second leg component along the longitudinal axis. In certain embodiments, the base has a first surface structured to form a complementary fit relative to the portion of a patient's anatomy. In certain embodiments, the method includes coupling the leg components of at least one leg with a spring. In certain embodiments, the method includes coupling the leg components with a slot and protrusion.

Disclosed herein are systems, devices, and methods for guiding surgical instruments to prepare a bone surface for joint reconstruction or repair. Example embodiments include a leg with a first component linearly repositionable relative to a second leg component, means for retaining an orthopedic cutting or impacting device, wherein movement of the orthopedic device repositions the first leg component relative to the second leg component, and means for coupling the leg to a specific portion of a patient's anatomy.

Also disclosed are methods for adjusting the relative position of components within a surgical guide. In a first step, a leg of the guide is linearly positioned so that its distal end is a predetermined first distance from an upper housing of the guide, which supports a surgical preparation device (e.g., reamer). In another step, the housing is raised or lowered with respect to the distal end of the leg without moving that distal end. The distal end of the leg may have a patient-matched portion that corresponds to a patient's acetabulum or other joint or anatomical area.

As discussed above, other examples of the systems, devices and methods for guiding surgical instruments to prepare a bone surface for joint reconstruction or repair include a surgical guide that comprises a base, a guide hub and a positive depth stop. The base has a first surface structured to fit in a predetermined configuration relative to a portion of a patient's anatomy. The guide hub is coupled to the base and has an aperture for guiding an orthopedic resurfacing device, such as a cutting or impacting device. The guide hub also has a solid interface configured to mate with a depth stop to limit movement of the device relative to the guide hub. The depth stop can be provided with or as part of the guide, for example, it could be integrated with the preparation device, or it may be provided with the surfacing device. The first surface of the base is structured to form a complementary fit to the portion of the patient's anatomy. The first surface may mate with a defect, such as a deteriorated region, in the patient's anatomy. In certain embodiments, the first surface protects a portion of the patient's bone from the orthopedic cutting or impacting device. In certain embodiments the predetermined configuration of the first surface is based on topography data indicative of the portion of the patient's anatomy.

Systems, devices, and methods described herein provide surgical guides with a positive depth stop to prevent excessive reaming by physically limiting the reaming depth. The depth stop can be coupled to the orthopedic cutting or impacting device. In certain embodiments, the depth stop is repositionable relative to the orthopedic cutting or impacting device. The depth stop may also be releasably attached to the orthopedic cutting or impacting device. The depth stop may be a sleeve, latch, wedge, or cone.

Systems, devices, and methods described herein provide surgical guides with guide hubs for guiding an orthopedic preparation device, such as a reamer, in a predetermined orientation. In certain embodiments, the predetermined orientation is based on a target abduction or anteversion angle determined in pre-operative plan. The hubs include an aperture that mates with the stem or rod of the orthopedic device to guide the orthopedic device. The hubs may also include a lateral opening for positioning a reamer within the aperture. The lateral opening may also allow a surgeon to see and monitor the surgical site during the procedure. The guide hub has a height that may be determined from topography data indicative of the patient's anatomy. The guide hub is coupled to the base. Systems, devices, and methods are provided for releasably coupling the hub to the base, for example, with a fastener. In other embodiments the guide hub is fixedly attached to the base, and may be unitary with the base.

Further disclosed herein are systems for guiding surgical instruments to prepare a bone surface for joint reconstruction. Examples include a base, guide hub, and depth stop. The base has a first surface structured to fit in a predetermined configuration relative to a portion of a patient's anatomy. The first surface preferably forms a complementary fit with the portion of the patient's anatomy. The guide hub is coupled to the base and has an aperture for guiding an orthopedic cutting or impacting device. The depth stop is structured to mate with the guide hub to limit movement of the orthopedic preparation device relative to the guide hub. In certain embodiments, the depth stop is coupled to the orthopedic cutting or impacting device, while in certain embodiments the depth stop is coupled to the guide hub or base. The orthopedic cutting or impacting device is typically one of a reamer, impactor, drill, or resurfacing tool.

Disclosed herein are methods of making an orthopedic surgical guide. In certain example methods, the steps include collecting topography data of a portion of a patient's anatomy, creating a model of the patient's anatomy from the data, determining a final depth for an orthopedic reaming or impacting device relative to the model, forming a base having a first surface structured to form a complementary fit relative to the portion of a patient's anatomy, forming a guide hub having an aperture for guiding an orthopedic cutting or impacting device, and forming a depth stop. The method may further include steps of determining an insertion angle for the orthopedic reaming or impacting device, and forming an inner surface of the aperture that corresponds to the determined insertion angle. In certain embodiments, the methods include forming the guide hub with a height based on the determined final depth of the orthopedic reaming or impacting device.

Disclosed herein are methods for using an orthopedic surgical guide by providing a patient-matched base and a depth stop to limit or otherwise direct the depth to which the cutting or impacting device can extend. Example implementations are provided by a surgical system having a base, a hub, and a depth stop. The base has a first surface structured to fit in a predetermined configuration relative to a portion of a patient's anatomy. The guide hub is coupled to the base and has an aperture for guiding an orthopedic cutting or impacting device and a solid interface configured to mate with the depth stop to limit movement of the device relative to the guide hub. Further steps may include placing the first surface of the base in the predetermined configuration on the portion of the patient's anatomy, placing the orthopedic cutting or impacting device within the aperture of the guide hub, and sliding the orthopedic cutting or impacting device until the depth stop couples with the solid interface of the hub. The method may also include placing the guide hub on the base and placing the depth stop on the orthopedic cutting or impacting device.

Disclosed herein is a surgical guide with a base, guide means, and depth stop means. The base has an interface means that forms a complementary fit relative to a portion of a patient's anatomy. The guide means is coupled to the base and guides an orthopedic cutting or impacting device. The depth stop means constrains the movement of the orthopedic cutting or impacting device.

Variations and modifications will occur to those of skill in the art after reviewing this disclosure. The foregoing features and aspects may be implemented, in any combination and subcombinations (including multiple dependent combinations and subcombinations), with one or more other features described herein. The various features described or illustrated above, including any components thereof, may be combined or integrated in other systems. Moreover, certain features may be omitted or not implemented.

### BRIEF DESCRIPTION OF THE DRAWINGS

The foregoing and other objects and advantages will be apparent upon consideration of the following detailed description, taken in conjunction with the accompanying drawings, in which like reference characters refer to like parts throughout, and in which:

FIGS. 1A and 1B show perspective views of an illustrative surgical guide with moveable legs for guiding a surgical preparation device;

FIGS. 2A-2C show various perspective and side elevation views of an illustrative surgical guide with a reamer at an acetabular joint;

FIGS. 3A and 3B show side elevation views of an illustrative surgical guide in a tilted position;

FIGS. 4A and 4B show perspective views of an illustrative surgical guide with four legs;

FIGS. 5A and 5B show side elevation views of an illustrative surgical guide with one leg;

FIGS. 6A and 6B show various side elevation and perspective views of an illustrative surgical guide with a patient-matched pedestal;

FIGS. 7A and 7B show side elevation views of an illustrative surgical guide configured to place a prosthetic cup at an acetabular joint;

FIGS. 8A-8C show schematic cross-sectional views of an illustrative drill guide for placing a surgical guide;

FIGS. 9-14 show various illustrative mechanisms for repositioning the legs of a surgical guide;

FIGS. 15-20 show schematic views of various illustrative mechanisms to provide leg motion;

FIG. 21 shows an illustrative flow chart for making a surgical guide;

FIGS. 22A-22E show various perspective and cross-sectional views of an illustrative orthopedic surgical guide having a depth stop for use with an orthopedic surgical preparation device, such as a reamer;

FIG. 23 shows a cross-sectional view of an illustrative surgical guide with an angled aperture for guiding a reamer or impactor;

FIGS. 24A-24C show cross-sectional views of an illustrative orthopedic surgical guide with a widened aperture;

FIG. 25 shows a cross-sectional view of an illustrative orthopedic guide with an aperture structured to guide the base of an orthopedic preparation device;

FIG. 26 shows a cross-sectional view of an illustrative orthopedic guide with a surface that protects a portion of a bone surface from reaming or other reshaping procedures;

FIG. 27 shows a cross-sectional view of an illustrative orthopedic guide with a detachable hub;

FIG. 28 shows a cross-sectional view of an illustrative hub and base of an orthopedic surgical guide;

FIG. 29 shows a cross-sectional view of an illustrative orthopedic guide with a hub that is coupled to a base with screws;

FIG. 30 shows a cross-sectional view of an illustrative orthopedic guide with a unitary base and hub;

FIG. 31 shows a schematic view of an illustrative sleeve depth stop for guiding a surgical preparation device;

FIGS. 32A-32B show schematic views of an illustrative latch depth stop;

FIG. 33 shows a schematic view of an illustrative ball latch depth stop;

FIG. 34 shows a schematic view of an illustrative wedge depth stop;

FIG. 35 shows a perspective view of an illustrative orthopedic surgical guide that encloses a surgical preparation device;

FIG. 36 shows a cross-sectional view of an illustrative orthopedic surgical guide with an impactor and acetabular shell;

FIG. 37 shows an illustrative flow chart for making an orthopedic surgical guide;

FIGS. 38A and 38B show side elevation views of an illustrative surgical guide with moveable legs for guiding a surgical preparation device and depth stops;

FIGS. 39A and 39B show side elevation views of an illustrative surgical guide with moveable legs for guiding a surgical preparation device and a depth stop for use with an orthopedic surgical preparation device, such as a reamer; and

FIG. 40 shows a cross-sectional view of an illustrative surgical guide with moveable legs for guiding a surgical preparation device and a depth stop for use with an orthopedic surgical preparation device, such as a reamer.

### DETAILED DESCRIPTION

To provide an overall understanding of the systems, devices, and methods described herein, certain illustrative embodiments will be described. Although the embodiments and features described herein are specifically described for use in connection with orthopedic hip procedures, it will be understood that all the components, connection mechanisms, adjustable systems, manufacturing methods, and other features outlined below may be combined with one another in any suitable manner and may be adapted and applied to devices and implants to be used in other surgical procedures, including, but not limited to hip arthroplasty, knee arthroplasty, spine arthroplasty, cranio-maxillofacial surgical procedures, shoulder arthroplasty, as well as foot, ankle, hand, and other extremity procedures.

Disclosed herein are systems, devices, and methods for guiding surgical instruments to prepare a bone surface for joint reconstruction or repair. The systems, devices, and methods disclosed herein incorporates features designed to limit movement of the orthopedic preparation device and thereby allow proper bone shaping of the joint at the proper depth, angle, and diameter to enable successful joint reconstruction and fixation of prosthetic implants. Some example devices and systems include a surgical guide system having a base and at least one linearly repositionable leg that provides tactile depth feedback and a limiting depth stop for an orthopedic preparation device, such as a reamer. In certain embodiments, the structure of the guide is planned pre-operatively to form a patient-matched design that appropriately accounts for individual patient anatomy. The guide may include a surface, such as a distal end, that forms a complementary fit with a specific portion of a patient's anatomy. The surface may be based on topographical data, such as MRI, X-ray, CT or CAT scan, or ultrasound images, of the patient's anatomy. For example, a reamer may be guided in a pre-planned insertion angle and depth relative to the surfaces of the joint, such as an acetabulum. Further disclosed herein are methods of making and using orthopedic surgical guides. The guide has numerous advantages that will be described herein.

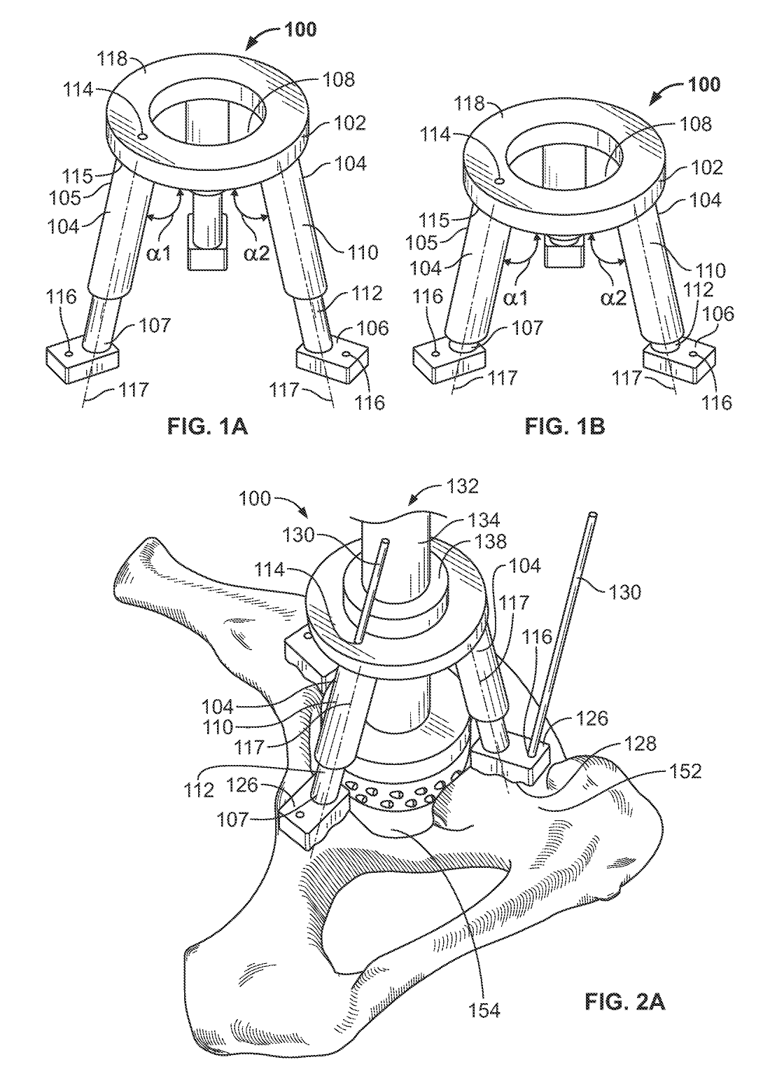

FIGS. 1A and 1B show an illustrative surgical guide for use with an orthopedic surgical preparation device, such as a reamer, impactor, or drill. The guide 100 shown in FIGS. 1A and 1B has a semi-rigid orientation with a retaining hub for a surgical preparation device that can be repositioned along at least one axis. The guide 100 also has a predetermined positive depth stop to define the reaming depth and prevent over-reaming.

The guide 100 has a housing, such as retaining hub 102, structured to couple with and retain an orthopedic surgical preparation device, such as a reamer, impactor, or drill. For example, the hub 102 has a support surface, such as aperture 108, that mates with a surgical preparation device, as will be further described herein. In alternative implementations, the support surface for retaining a surgical preparation device is a slot or crevice. The guide 100 has legs 104 coupled to the retaining hub 102. In certain embodiments, the legs are fixedly coupled to the hub 102. The legs 104 may also be removable from the hub 102. Preferably, the legs 104 are structured to orient the hub 102 and surgical preparation device in a predetermined orientation. The legs 104 comprise an upper portion 110 and a lower portion 112, which are linearly repositionable relative to each other along their common longitudinal axis 117. In certain embodiments, as shown in FIGS. 1A and 1B, the guide 100 includes a plurality of legs 104. A base or foot 106 is coupled to lower portion 112 at the distal end 107 of the leg 104. The foot 106 serves as a base for the guide 100 to allow the guide 100 to securely interface with the bone or tissue of a patient. In certain embodiments, the foot 106 has an aperture 116 for receiving a fastener such as a pin. The hub 102 may also have an aperture 114 for receiving a fastener, such as a bone pin. In certain embodiments, the hub aperture 114 extends through the leg 104 and foot 106.

The upper portion 110 of the leg and lower portion 112 of the leg 104 are repositionable relative to each other to allow the hub 102 to move up and down for repositioning an orthopedic preparation device at a surgical site. FIG. 1B shows the leg 104 in a lowered or compressed position, which allows a surgical preparation device to move about the surgical site. The upper portion 110 is coupled to the hub 102 so that movement of the leg 104 vertically repositions hub 102 with respect to the patient's anatomy. The distal end 107 of the leg 104 is initially placed at a predetermined distance from the housing or hub 102 of the guide. As the hub 102 is lowered, the distal end 107 of the leg remains fixed, and the hub 102 is brought closer to the distal end 107, as seen in FIG. 1B. Similarly, when the hub 102 is raised, the hub 102 is repositioned to a further distance from the fixed distal end 107. The movement of upper portion 110 relative to lower portion 112 has a limited operational range of motion. In certain embodiments the movement mechanism of the leg 104 includes a spring or slot to control motion, provide tactile feedback, and limit the reaming depth, as will be described herein. In certain embodiments, the angle of attachment α1, α2 of the legs 104 relative to the hub 102 is adjustable, for example, by a latch, ratchet, or other mechanical connection between the proximal end 105 of the leg and the lower rim 115 of the hub 102. In alternative embodiments, the legs 104 are in a fixed orientation relative to the hub 102.

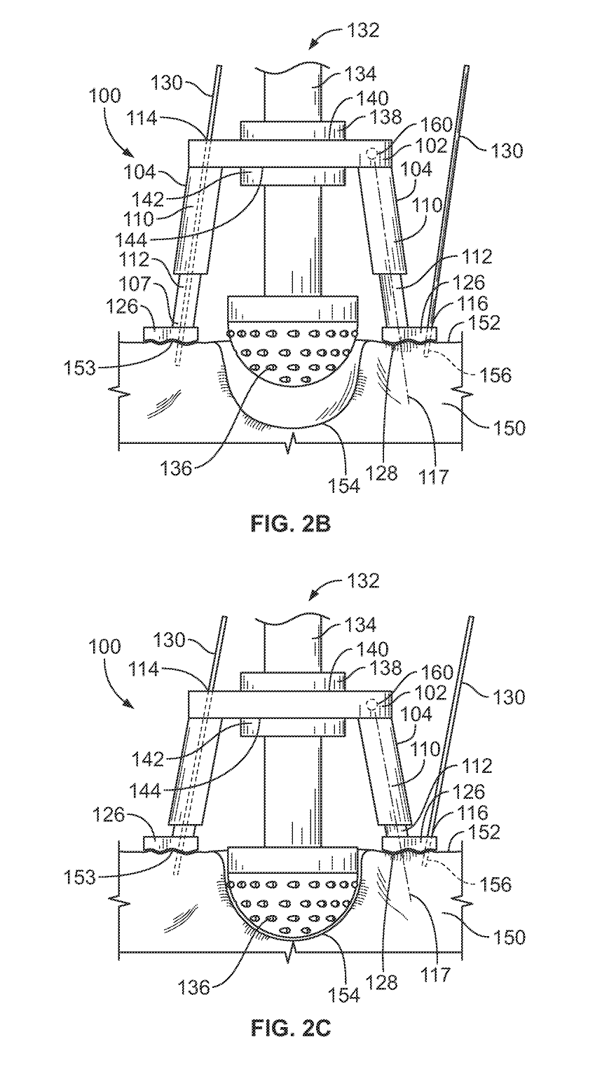

FIGS. 2A-2C show an illustrative surgical guide 100 placed with a reamer 132 positioned near an acetabulum 154. The guide 100 orients the reamer 132 and provides tactile feedback when moving the reamer 132. The legs 104 of the guide 100 provide a limited operational range of movement along the longitudinal axis 117 of the legs 104 and a positive depth stop for the reaming process. The guide is secured to the bone or tissue by one or more bone pins 130, which fix the distal end 107 of the legs 104 relative to the surgical site, such as acetabulum 154. As shown in FIGS. 2A-2C the rod 134 of the reamer 132 is placed within the aperture 108 of the hub 102 and held in place by an upper retaining sleeve 138 and a lower retaining sleeve 142. In certain embodiments, the diameter of the rod 134 is substantially similar to the diameter of the aperture 108 to form a tight fit between the rod 134 and aperture 108. In alternative embodiments, the diameter of the aperture 108 is larger than the diameter of the rod 134 to allow flexibility in positioning the reamer 132 or other surgical device.

As seen in FIGS. 2B and 2C, the retaining sleeves 138 and 142 are tightly coupled to the rod 134 of the reamer 132 to securely fix the rod 134 in the hub 102 between the retaining sleeves 138 and 142. The retaining sleeves 138 and 142 have contact surfaces 140 and 144 respectively that contact the hub 102 to retain the reamer 132 in place. In certain embodiments, only an upper retaining sleeve 138 is used. When only upper retaining sleeve 138 is used, the rod 134 of the reamer may freely slide upward within the aperture 108, but downward movement is limited by the coupling of the upper retaining sleeve with the hub 102. In alternative embodiments, a retaining sleeve is provided with the orthopedic surgical preparation device. In certain embodiments, the hub 102 retains the reamer 132 directly without a retaining sleeve. Other means of retaining the orthopedic surgical preparation device may also be used, including, but not limited to, adhesives, friction fit, clamps, and latches.

In FIGS. 2A and 2B, the reamer 132 is in a raised position. The upper retaining ring 138 couples the rod 134 to the hub 102, which is coupled to the leg 104. The hub 102 is positioned at a predetermined distance from the foot 126 at the distal end 107 of the leg 104, which is secured to the bone 150. The hub 102 is repositionable relative to the distal end 107 of the leg 104 through the movement of the upper portion 110 and lower portion 112 of the leg. When a sufficient force is applied along the reamer 132 downwards toward the acetabulum 154, the upper portion 110 of the leg 104 moves relative to the lower portion 112 of the leg 104 along axis 117 to move the cutting portion 136 of the reamer 132 into contact with the acetabulum 154. As the cutting portion 136 progresses deeper within acetabulum 154, the legs 104 preferably provide increased resistance to the downward motion of the reamer 132, for example, by compressing a spring along axis 117. This resistive force provides the surgeon with tactile feedback relative to the depth of the reamer 132 within the acetabulum 154. The depth of the reaming is controlled by operational range of motion between upper portion 110 and lower portion 112, which provides a positive depth stop for the reaming process at the limit of the allowed motion.

In the embodiment of FIGS. 2A-2C, each leg 104 of the guide 100 include a foot 126, similar to foot 106, coupled to the lower portion 112. The guide 100 may be fixed in place by a bone pin 130, which extends through the aperture 116 of the foot 126 or through the aperture 114 of the hub 102 and into the bone 150. In certain embodiments, a plurality of pins 130 may be provided and used to secure the guide 100 to the bone 150.

In contrast to foot 104, the distal face of the foot 126 has a bone interface surface 128 that forms a complementary fit 153 with the surface 152 of the bone 150. The bone interface surface 128 fits in a unique predetermined position on the patient's anatomy to appropriately orient the guide and the reamer according to a pre-operative plan. Appropriate positioning of the surface 128 on the patient properly aligns the guide 100. As seen, for example, in FIGS. 2B and 2C, the bone interface surface 128 is contoured to conform to the specific anatomical features of the patient. The surface 126 of the foot 126 may have portions that fit to anatomical landmarks, portions of the acetabulum or acetabulum rim, defects, or other identifiable features to form complementary fit 153 to rapidly and securely place the guide. In certain embodiments those identifiable features are determined based on topographical data indicative of the patient's anatomy, and the data is then used to form the bone interface surface 128. For example, MRI, X-ray, CT, or ultrasound images may be taken of the patient's anatomy to form a model, which is then used as a reference surface for creating a complimentary interface surface 128. In certain embodiments, the model is a physical model. Alternatively, the model may be a computer model.

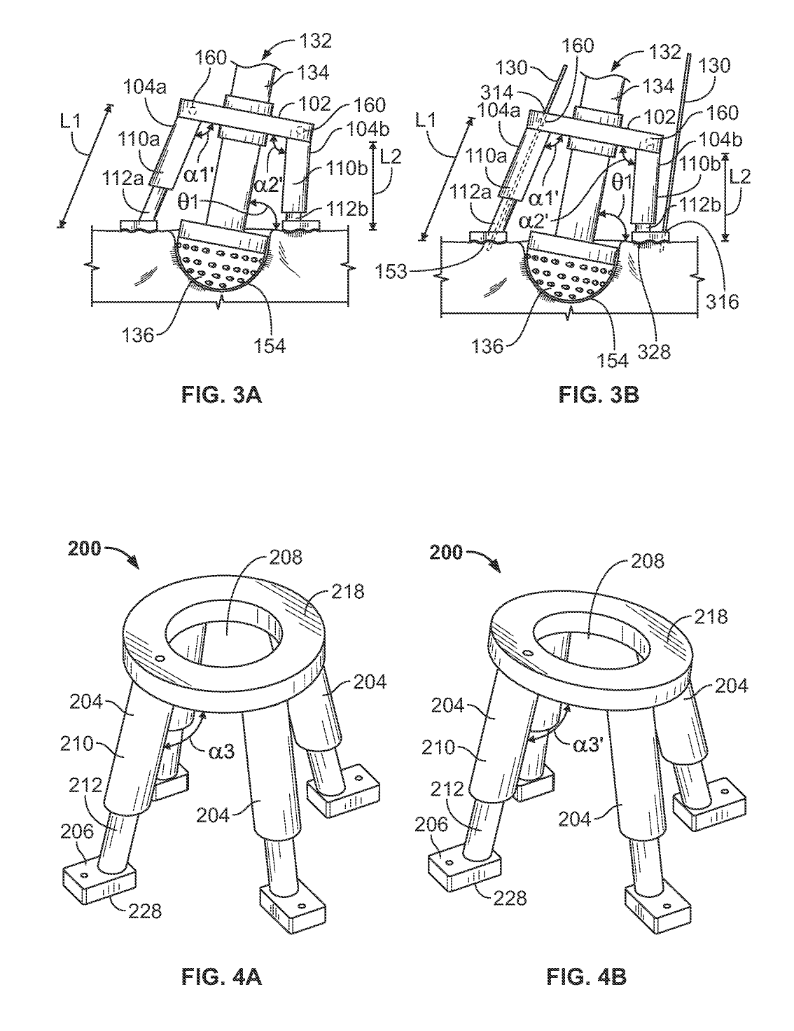

The surgical guide 100 may be adapted to guide a reamer 132 or other surgical device in a variety of orientations. For example, FIG. 3 depicts the surgical guide 100 in a tilted position. The pre-operative plan may define desired abduction and anteversion angles for the placement of a prosthetic acetabular cup, which, in turn, determines the angle θ1 at which the reamer will be inserted. The abduction and anteversion angles may be determined from patient data, such as an MRI, X-ray, CT, or ultrasound images. In certain embodiments, abduction and anteversion angles are based on predetermined target angles, such as a 45° abduction angle and a 15° anteversion angle. The hub 102 may be tilted at any appropriate angle as required for the desired abduction and anteversion angles. The legs 104a, 104b may be adjustable, as will be described herein, to accommodate the tilted angle of the guide 100. Alternatively, legs may be provided that have different lengths or that couple to the hub 102 at different angles.

The legs 104a, 104b may be adjustable, as will be described herein, to change the inclination of the hub and the corresponding insertion angle θ1 of the reamer and the reaming depth. For example, in certain embodiments the lengths “L1” and “L2” of the legs 104a and 104b are adjustable. In certain embodiments the angle a, such as α1′ and α2′, between the legs 104a, 104b and the hub 118 is adjustable by a pivot or rotation mechanism. For example, the legs 104a, 104b may attach to the rim with a hinge connection 160 that provides adjustability of the angle a between the legs 104a, 104b and the hub 118. In certain embodiments, the angle a is a fixed angle. The lengths “L1,” “L2” and angles α1′, α2′ of the legs 104a104b relative to the hub may be determined in a pre-operative plan. In certain embodiments these parameters may be adjusted during the procedure to allow the surgeon to account for surgical variability. In certain embodiments, legs 104a, 104b may be adjustable within a specified range to provide a target range for the insertion angle, such as a 30-50° abduction (inclination) angle and a 5-25° anteversion angle. The ability to adjust the legs 104a, 104b allows the surgeon to intra-operatively determine the most appropriate reaming and cup placement angle. The ability to adjust the legs 104a, 104b may also allow the legs 104a, 104b or guide 100 to be used in multiple procedures or multiple steps (e.g., reaming, drilling, etc.) of a given procedure by adjusting the legs 104a, 104b, as necessary.

As indicated above, the legs, such as legs 104a, 104b, may be detachable from the hub 102. For example, the guide 100 may be provided with a set of legs having a standard set of dimensions such as height, range of motion, and attachment angle. The legs of the guide 100 may be chosen from the standard set to appropriately orient the guide 100 and the surgical preparation device in accordance with the pre-operative plan based on patient data. In certain embodiments, a set of legs is provided as a kit of legs of different lengths or heights for use with a range of abduction and anteversion angles. The legs may be detachable, removable, and replaceable, for example, at connection 160. In certain implementations, the guide 100 is provided with a standard set of legs that can be replaced or substituted with legs from a kit.

The surgical guide system may comprise any number of legs in order to appropriately position and guide a surgical preparation device. For example, as shown in FIGS. 1-3, the guide has three legs. In other embodiments, such as those depicted in FIGS. 4A and 4B, the surgical guide system has four legs. Guide 200 is similar to guide 100 in that it has a hub 218 with an aperture 208 for holding a surgical preparation device. The guide 200 has legs 204 with an upper portion 210 and a lower portion 212 coupled with a foot 206. The additional legs allow increased freedom in refining or adjusting the guiding orientation for the surgical preparation device. For example, FIG. 4B shows the guide 200 in a tilted position, with angle α3 adjusted to angle α3′.

In certain embodiments, the legs are provided with a visual indicator of the height, abduction angle, and anteversion angle. For example, a visual indicator may be a color-coded bit or extension piece protruding through the top of the leg near the hub. Increasing the quantity of legs can provide increased degrees of freedom for adjusting the hub. For example, with higher numbers of legs, the abduction and anteversion (inclination and version) angles of the hub relative to the patient's anatomy can be adjusted independently.

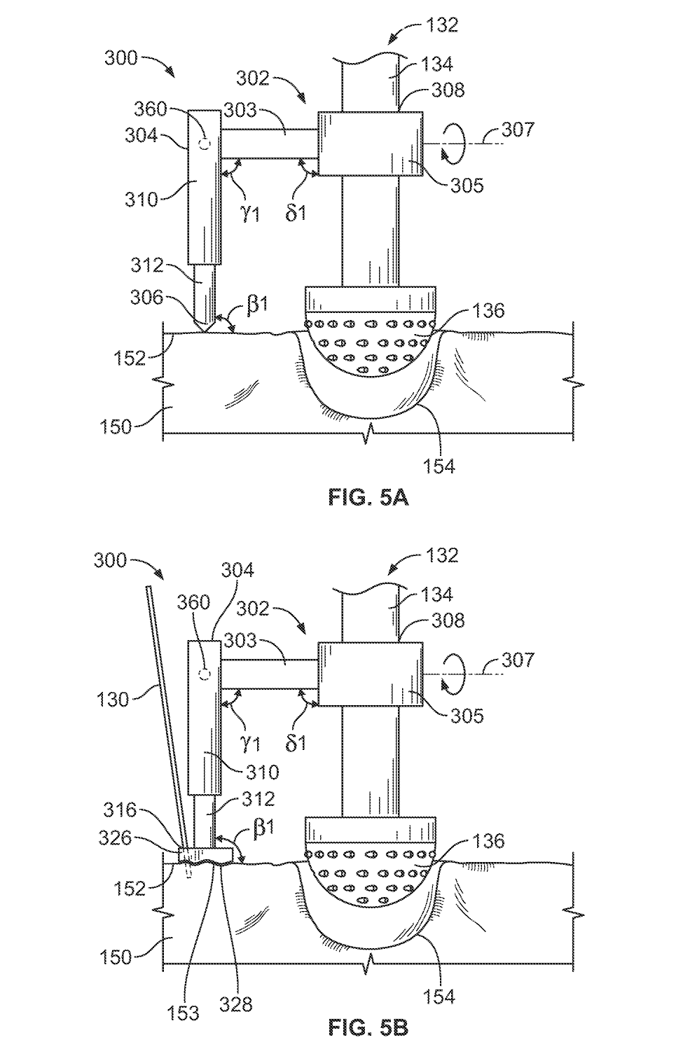

In other embodiments the guide is provided with a single leg. For example, FIG. 5A shows an illustrative guide 300 with a leg 304 adapted to retain and guide reamer 132. The leg 304 includes an upper portion 310 and a lower portion 312, similar to upper portion 110 and lower portion 112 of leg 104, that are adjustably positionable relative to each other. A base in the form of a foot 306 is coupled to the lower part 312 of the leg 304. The hub 302 of the guide 300 has a retainer 305 and an arm 303. The arm 303 couples the leg 304 to the retainer 305. The retainer 305 includes an aperture 308 adapted to retain the rod 134 of the reamer 132. The foot 306 is adapted to couple to the bone surface 152 of the bone 150. In certain embodiments the foot 306 has the form of a pin or screw that is inserted directly into the bone 150. FIG. 5B depicts a similar embodiment with a foot 326 that has a bone interface surface 328 that forms a complementary fit 153 with the surface 152 of the bone 150. As shown, the guide 300 may also be secured with pin 130 inserted through one or more apertures 316 and into the bone 150. In certain embodiments, the pin 130 is inserted through the leg 304.

The rod 134 of the reamer 132 is fixedly coupled within the aperture 308 of the retainer 305, and may be retained with retaining sleeves, as discussed with previously described embodiments. The leg 304 provides restricted range of motion for the reamer 132. As the reamer 132 is pushed downward toward the acetabulum 154, the upper portion 110 and lower portion 112 of leg 104 move relative to each to allow the reamer 132 to contact the acetabulum. The leg 304 provides resistive feedback to the motion of the reamer 132 during the procedure. Additionally, the restricted range of motion of leg 304 provides a positive depth stop to limit the reaming depth.

The guide 300 can be adjusted to appropriately guide the reamer 132 into a desired reaming orientation of the acetabulum 154. For example, the pre-operative plan may define an angle β1 between the leg 304 and the bone surface 152. The pre-operative plan also define an angle γ1 between the leg 304 and the arm 303. Further, the pre-operative plan may define an angle δ1 between the arm 303 and the retainer 305. In certain embodiments the angular position of the reamer 132 may be adjusted by rotation about the axis 307 through the arm 303.

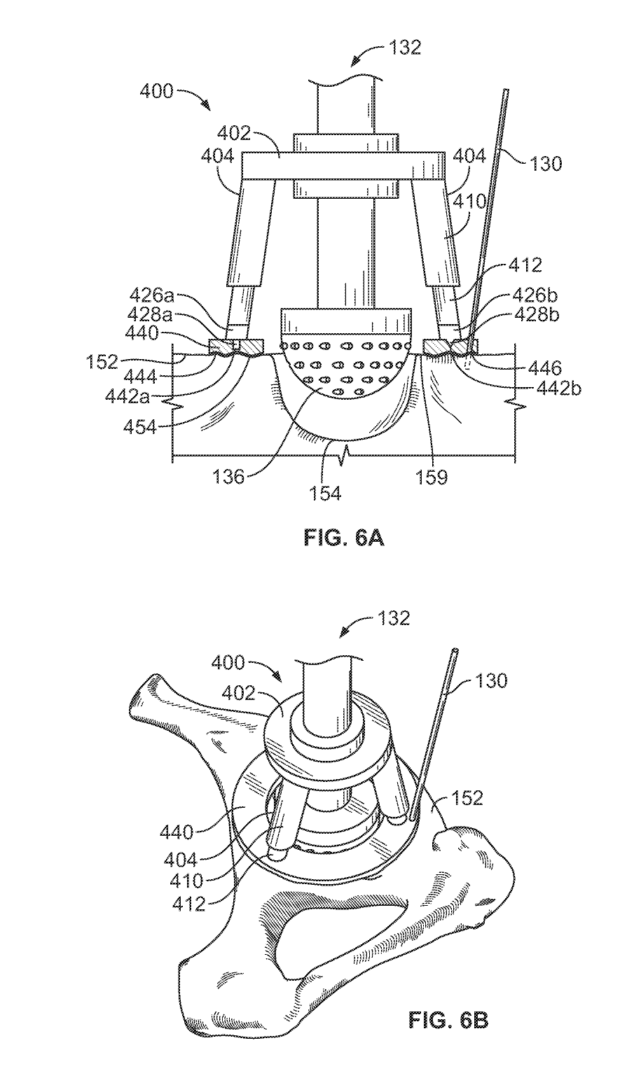

FIGS. 6A and 6B depict an example surgical guide 400 that mates with a pedestal 440. The pedestal 440 includes a bone interface surface 444 adapted to form a complementary fit 454 with the bone surface 152 of the patient. The pedestal is secured to the bone by means of a pin 130 inserted through the pin hole 446. As shown in FIG. 6B, the pedestal 440 encloses the rim 159 of the acetabulum 154. In certain embodiments, the pedestal partially surrounds the rim 159 of the acetabulum.

As depicted, the pedestal 440 includes one or more receiving holes 442a or 442b adapted to receive a respective locking feature or peg 428a or 428b of the foot 426a or 426a. In certain embodiments the locking feature, such as peg 428a, fits only in one receiving hole, such as 442a, to properly orient the guide 400 at the acetabulum 154. Similarly, locking peg 428b fits only in hole 442b. The shape and position of the receiving holes 442a and 442b and locking pegs 428a and 428b allow the surgeon to rapidly and easily orient the guide 400 and position the reamer 132 to prepare the acetabulum surface 154. In certain embodiments, the receiving holes 442a and 442b and locking pegs 428a and 428b have a substantially similar shape. Preferably, the guide 400 is removable from the pedestal 440. In certain implementations the guide 400 is fixedly attached to the pedestal 440. In alternative implementations, the guide 400 and pedestal 440 are integrally formed. The reamer 132 is attached to the hub 402. The upper portion 410 of the leg 404 linearly moves relative to the lower portion 412 of the leg 404 within a restricted operational range of motion to allow the reamer to contact the acetabulum 154, while providing both tactile feedback and a reaming depth stop.

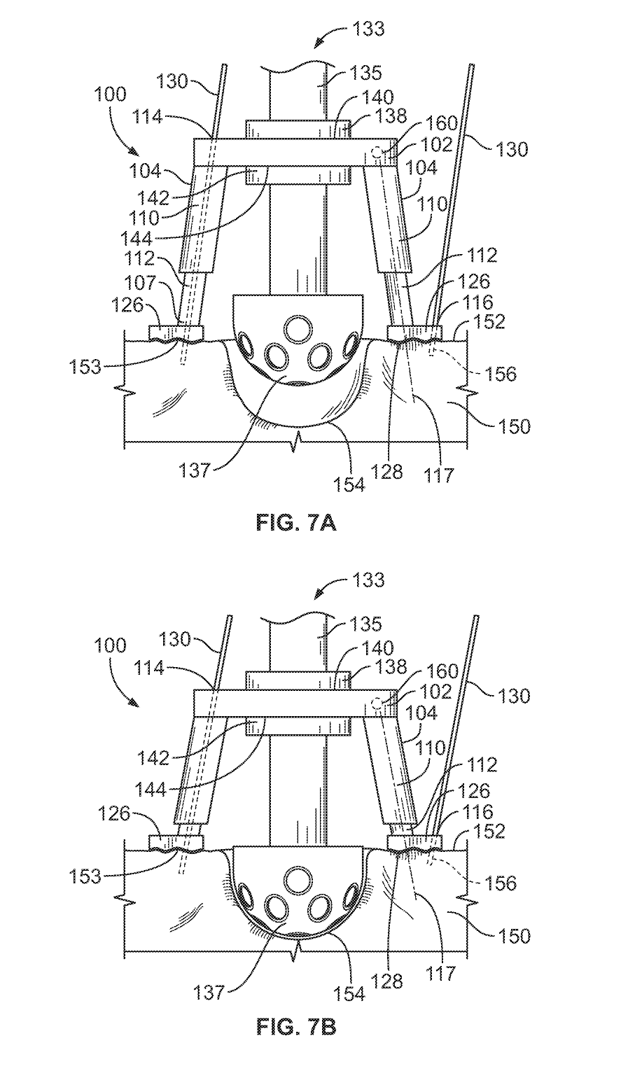

While primarily depicted with a reamer for illustrative purposes, the surgical guide (e.g. guide 100, 200, 300, 400) may also be used with other surgical preparation devices, including, but not limited to, a reamer and a drill. The guide can be used to accurately place an acetabular implant in a specific orientation. For example, FIGS. 7A-7B depict surgical guide 100 configured with an impactor 133 for placing a prosthetic cup 137 at the acetabular joint 154. FIG. 7A depicts the impactor 133 in a raised position, while FIG. 7B depicts the impactor 133 in a lowered position with the acetabular cup 137 positioned in the acetabulum 154. The guide 100 provides similar guiding functionality to the impactor 133 as described when used with the reamer 132. For example, the rod 135 of the impactor 133 is retained within the aperture 108 of the guide 100. The impactor 133 can be guided at an appropriate angle for placement of the cup 137. As previously described, the height, angle, and depth stop of the guide 100 and legs 104 are adjustable to provide accurate placement of the cup 137 and adjustability before and during the procedure, as required by the surgeon.

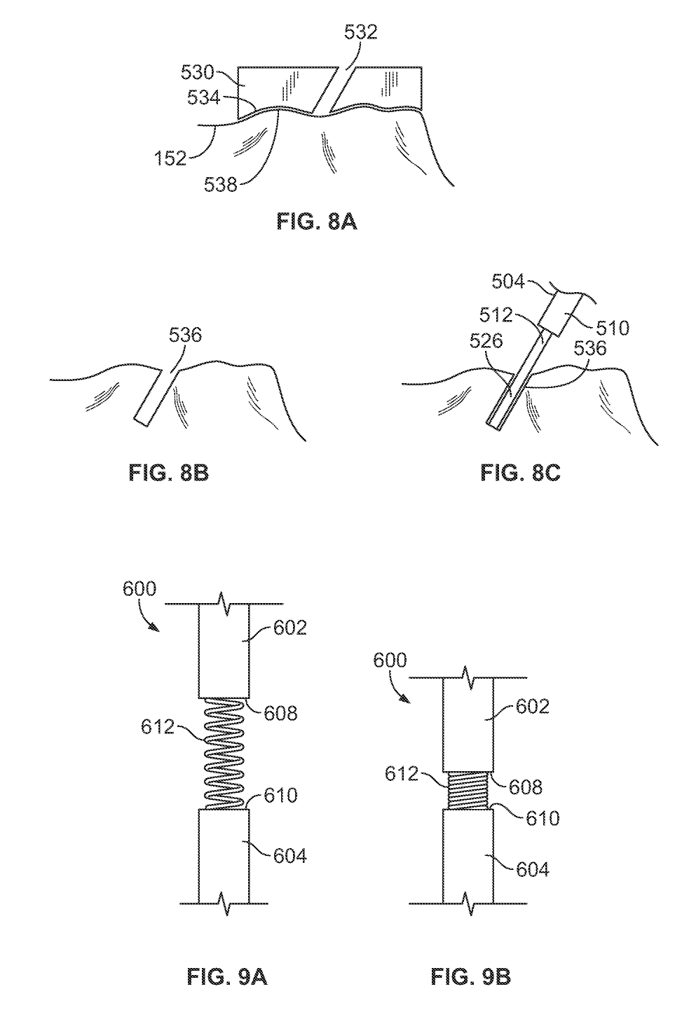

In use, it may be advantageous to pre-drill the patient anatomy to provide holes for installation of the guide. FIGS. 8A-8C illustrate a drill guide 530 used to drill placement holes for the legs of a surgical guide. The drill guide 530 includes a bone interface surface 534, similar to surfaces 128 and 444, that forms a unique, complementary fit 538 with a specific portion of the bone surface 152 of the patient. The drill guide 530 includes a guide hole 532 for drilling a hole 536 in the bone surface 152. The placement and orientation of the guide hole 532 may be determined in a pre-operative plan for the specific patient. After placing the drill guide 530 on the bone surface 152, a drill is inserted through the guide hole 532 to form a hole 536 in the bone surface 152. After the hole 536 is formed, the foot 526 of a leg 504 is placed within and securely retained by hole 536. The leg 504 is part of a surgical guide, such as guide 100. Placement of the foot 526 in the drilled hole 536 properly aligns the guide and orthopedic surgical preparation device (e.g., reamer 132, impactor 133) for accurately preparing the acetabulum or placing the implant according to the pre-operative plan for the specific patient.

FIGS. 9-14 depict various legs for a surgical guide that provide linear repositionability, tactile feedback during movement, and a positive depth stop. FIGS. 9A and 9B show a leg 600 with an upper portion 602 coupled to a lower portion 604 with a spring 612. In certain embodiments, the spring 612 is a compression spring formed from a metal, such as spring steel. In alternative embodiments, the spring 612 is an elastomer. The spring 612 is disposed between the end 608 of the upper portion 602 and the end 610 of the lower portion 604. When the upper portion 602 and the lower portion 604 are brought closer together, for example, by movement of the orthopedic surgical preparation device (e.g., reamer 132, impactor 133), the spring 612 is compressed and exerts a force proportional to its change in length. As the spring 612 is further compressed, spring 612 exerts an increasingly greater resistive force on the upper portion 602 and the lower portion 604. This resistive force provides tactile feedback for the surgeon to guide the reaming process. In FIG. 8B, the ends 608 and 610 have fully compressed the spring 612 and reached the limit of their relative motion. This physical limit serves as a positive depth stop for the reaming process.

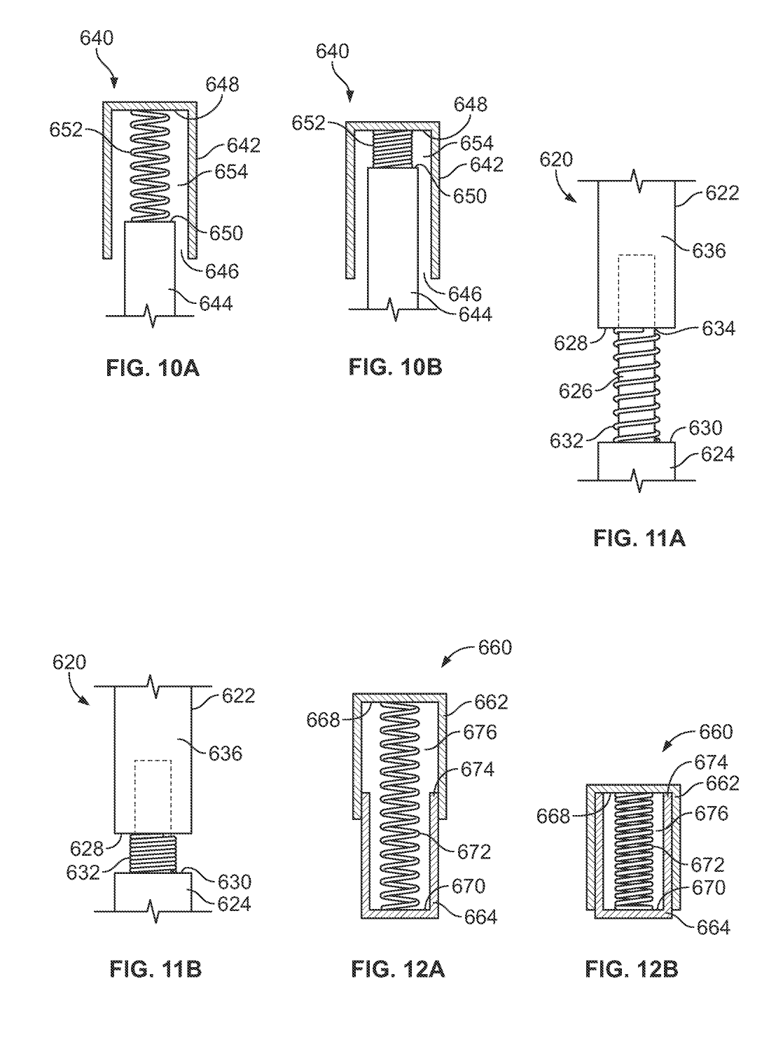

FIGS. 10A-10B depict an alternative embodiment of a leg 640 with a lower portion 644 and an upper portion 642. FIG. 10A shows the leg 640 in an extended position, while FIG. 10B shows the leg 640 in a compressed position. The upper portion includes an aperture 646 through which the lower portion 644 slides into the chamber 654 of the upper portion 642. The leg 640 includes a compression spring 652, similar to spring 612, disposed between the end 650 of the lower portion 644 and the inner end 648 of the upper portion 642 within the chamber 654. In certain embodiments, the spring 652 is a compression spring formed from a metal, such as spring steel. In alternative embodiments, the spring 652 is an elastomer. As the end 650 of the lower portion 644 is brought nearer to the end 648 of the upper portion 642, the spring 652 applies an increasingly resistive force to give the surgeon tactile feedback and a positive depth stop in the reaming process.

FIGS. 11A-11B depict an alternative embodiment of a leg 620 with a lower portion 624 having a column 626 that fits through an opening 634 into a hollow chamber 636 of the upper portion 622. The leg 620 includes a spring 632 disposed around the column 626 of the lower portion 624. In certain embodiments, the spring 632 is a compression spring formed from a metal, such as spring steel. In alternative embodiments, the spring 632 is an elastomer. The spring 632 is positioned between the end 628 of the upper portion 622 and the ledge 630 of the lower portion 624. As shown in FIG. 11B, as the upper portion 622 and lower portion 624 are brought closer together, the column 626 moves along the chamber 636 and the spring 632 is compressed between the end 628 of the upper portion 622 and the ledge 630 of the lower portion 624. As in the previous embodiments, the resistive force of the spring 632 during compression provides tactile feedback during movement of the orthopedic surgical preparation device (e.g. reamer 132, impactor 133). A positive depth stop to limit reaming depth is provided when the spring 632 is fully compressed.

FIGS. 12A-12B depict an alternative embodiment of a leg 660 having a lower portion 664 that slides within an upper portion 662. As shown, the upper portion 662 and lower portion 664 are substantially hollow. The interlocking of the upper portions 662 and 664 creates a chamber 676 in which a compression spring 672 is positioned. In certain embodiments, the spring 672 is a compression spring formed from a metal, such as spring steel. In alternative embodiments, the spring 672 is an elastomer. The spring 672 is positioned between the upper end 668 of the upper portion 662 and the lower end 670 of the lower portion 664. As the lower portion 664 moves within the chamber 676, the spring 672 provides a resistive force to the movement, which is limited by the full compression of the spring 672. The movement may also be limited when the upper end 674 of the lower portion 664 contacts the upper end 668 of the upper portion 662.

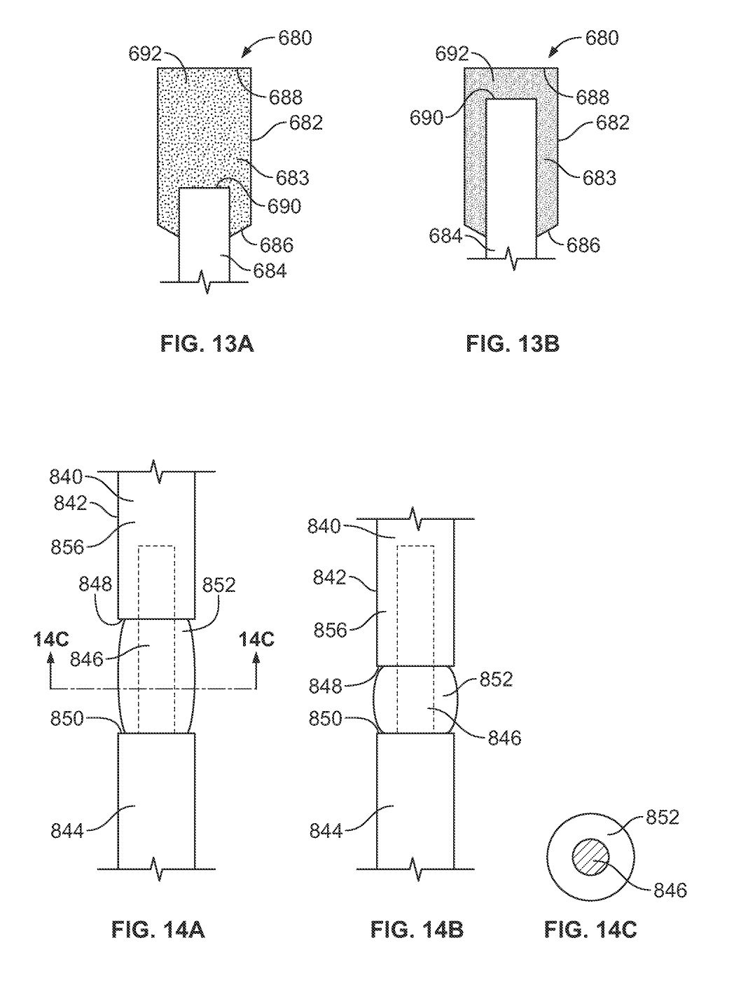

FIGS. 13A-13B depict an alternative spring mechanism for use in a leg of a surgical guide. Leg 680 includes an upper portion 682 and a lower portion 684. The upper portion 682 is substantially hollow with a chamber 683. The end 690 of the lower portion 684 fits within the chamber 683. The chamber 683 also includes a compressive material 692. In certain embodiments a seal 686 is provided between the upper portion 682 and the lower portion 684 to enclose the chamber 683. In alternative embodiments, the upper portion 682 and lower portion 684 are formed with a tight interface. In certain embodiments the compressive material 692 is one of an elastomer, foam, and gas. Similar to other springs, as the compressive material 692 is compressed, it provides a resistive force of the movement between of upper portion 682 to lower portion 684. The motion is limited by the full compression of the elastic material 692, as shown in FIG. 13B. The movement may also be limited when the end 690 of lower portion 684 reaches the end 688 of the upper portion 682.

FIGS. 14A-14C depict an embodiment of a leg 840 with a lower portion 844 having a column 846 that fits into a hollow chamber 856 of the upper portion 842. The leg 840 includes a ring 852 disposed around the column 846 of the lower portion 8444. FIG. 14C depicts a cross-sectional view through C-C’ of the ring 852 around column 846. In certain embodiments, the ring 852 is a formed from an elastomeric material, such as foam or other elastomeric polymer that provides compressive resistance. The ring 852 is positioned between the end 848 of the upper portion 842 and the ledge 850 of the lower portion 844. As shown in FIG. 14B, as the upper portion 842 and lower portion 844 are brought closer together, the column 846 moves along the chamber 856 and the ring 852 is compressed between the end 848 of the upper portion 842 and the ledge 850 of the lower portion 844. Similar to previous embodiments, the resistive force of the ring 852 during compression provides tactile feedback during movement of the reamer 132 or other orthopedic instruments, such as impactor 133. A positive depth stop to limit reaming depth is provided when the ring 852 is fully compressed.

The range of motion of the legs defines and limits the maximum reaming depth during the procedure. In certain embodiments the surgeon or technician may want flexibility in determining the reaming depth but may still want to have the safety of a positive stop mechanism to prevent excessive reaming. An adjustable depth mechanism may also be desirable for having one or more standard legs that can be used in multiple procedures or multiple steps of a single procedure. Adjustable legs may also be interchangeable with different hubs, feet, or drill guides, and may be part of a set of legs provided in a kit.

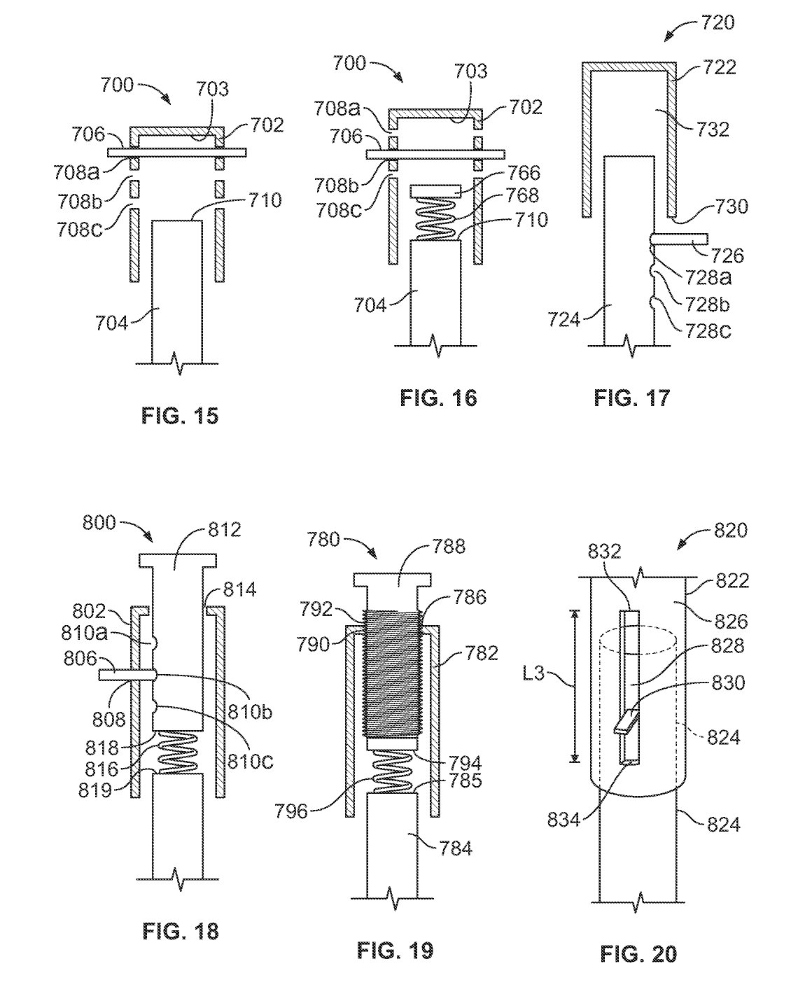

FIGS. 15-19 show example mechanisms for adjusting the range of movement of a leg. FIG. 15 depicts leg 700 having an upper portion 702 and a lower portion 704. The upper portion 702 includes stop pin holes 708a-c. A stop pin 706 may be inserted into any of the stop pin holes 708a-c to limit the movement of the lower portion 704 relative to the upper portion 702. In certain embodiments, as shown in FIG. 15, the stop pin holes 708a-c extend through the upper portion 702 of leg 700. FIG. 15 depicts stop pin 706 in stop pin hole 708a, but the pin may be placed in any stop pin hole 708a-c . Placement of the pin 706 in a different hole, for example 708c, would further restrict the range of motion. The pin 706 limits the relative movement between the upper portion 702 and lower portion 704 by restricting the movement of lower portion 704 toward the end 703 of the upper portion 702. The lower portion 704 may abut the pin 706, but cannot proceed further.

FIG. 16 depicts a leg 700 with a stop pin 706 and a spring 768. The spring 768, which includes stop piece 766, is disposed between the pin 706 and the lower portion 704. As the upper portion 702 and lower portion 704 are moved relative to each other, the stop piece 766 engages the pin 706. When engaged, the spring 768 provides resistive feedback and limits the relative motion of the upper portion 702 to lower portion 704.

Alternatively, as shown in FIG. 17, the lower portion 724 of a leg 720 may have stop pin holes 728a-c. A stop pin 726 is inserted into a stop pin hole 728A-C and as the lower portion 724 slides into the chamber 732 of the upper portion 722, the stop pin 726 will abut the end 730 of the upper portion 722 to limit the motion of the leg 720.

FIG. 18 depicts a leg 800 with an adjusting pin 812 coupled with a stop pin 806. The adjusting pin includes a plurality of stop pin holes 810a-c. The upper leg portion 802 of the leg 800 also includes a stop pin aperture 808. The stop pin 806 is placed through the aperture 808 and into a stop pin hole 810a-c to fix the adjusting pin 812 relative to the upper portion 802. A spring 816 is disposed between the end 818 of the adjusting pin 812 and the end 819 of the lower portion 804 of the leg 800. Placing the stop pin 806 in different stop pin holes 810A-C adjusts the position of the adjusting pin 812, and accordingly adjusts the height of the leg 800 and the reaming depth.

FIG. 19 shows an additional embodiment of a leg 780 with adjusting screw 788, The upper portion 782 includes an aperture 786 with threads 790 structured to engage the threads 792 of the screw 788. Turning the screw 788 adjusts the position of the screw within the upper portion 782 and accordingly adjusts the height of the leg 780. A spring 796 is disposed between the end 785 of the lower portion 784 and the end 794 of the screw 788 to provide resistive feedback during the procedure.

FIG. 20 shows an alternative embodiment for providing restricted movement of a leg 820. The leg 820 includes an upper portion 822 with a slot 828 and a lower portion 824 with a ledge 830. The lower portion 824 slides within the chamber 826 of the upper portion 822 of the leg 820. The ledge 830 of the lower portion 824 protrudes through the slot 828 of the upper portion 822. The movement of the upper portion 822 relative to the lower portion 824 is limited by the length “L3” of the slot 828, along which the ledge 830 slides. The motion is limited when the ledge 830 reaches upper end 832 or lower end 834 of the slot 828. In certain embodiments the ledge 830 is adjustably positionable on the lower portion 824 of the leg. In certain embodiments, the ledge 830 may be placed at different positions of the lower portion 824 of the leg 820 to adjust the height of the leg 820. For example, the ledge 830 may be a stop pin and the lower portion 824 may have a plurality of stop pin holes.

The terms “upper” and “lower” are used for exemplary purposed to describe the figures, however, the functionality of the upper and lower portions of legs can be provided with either the upper and lower portions of a leg. For example, although the legs of the guide have primarily been described with a lower portion of the leg sliding into an upper portion of the leg. In practice, the guide and leg may be configured so that an upper portion slides into a lower portion of the leg.

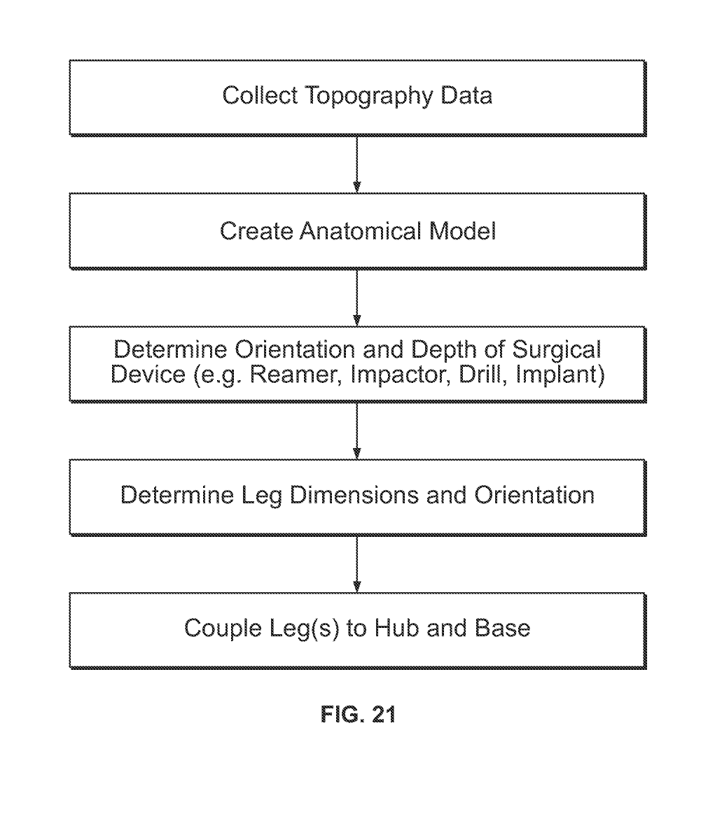

FIG. 21 describes a method of making a an orthopedic surgical guide for a specific patient. Initially, topography data of a portion of a patient's anatomy is taken, for example by imaging techniques including, but not limited to, MRI, X-ray, CT, and ultrasound. The data is then used to create a model of the patient's anatomy from the data. In certain embodiments, the model is a physical model or replica of the portion of the patient's anatomy. In alternative embodiments, the model is a computer model, such as a 3-dimensional image. The model is used to develop a pre-operative plan and determine the orientation and depth of the surgical preparation device, such as a reamer, impactor, or drill. Preferably, the plan defines abduction and anteversion angles for the placement of a prosthetic acetabular cup, which, in turn, determines the orientation of the surgical preparation device. The leg dimensions and orientation are chosen to properly orient the surgical preparation device. The legs are then coupled to the hub and base to form a complete surgical guide. In certain embodiments, the base includes a surface based on the topography data that forms a complementary fit with the specified portion of the patient's anatomy.

As discussed above, disclosed herein are systems, devices, and methods for guiding surgical instruments to prepare a bone surface for joint reconstruction or repair. The systems, devices, and methods disclosed herein incorporates features designed to limit movement of the orthopedic preparation device and thereby allow proper bone shaping of the joint at the proper depth, angle, and diameter to enable successful joint reconstruction and fixation of prosthetic implants. The example devices and systems above include a surgical guide system having a base and at least one linearly repositionable leg that provides tactile depth feedback and a limiting depth stop for an orthopedic preparation device, such as a reamer. Further example devices and systems, discussed below, include a surgical guide that operates with a positive depth stop for an orthopedic reaming or impacting device. The structure of the guide is planned pre-operatively to form a patient-matched design that appropriately accounts for individual patient anatomy. The devices and systems may include a surface that forms a complementary fit with a specific portion of a patient's anatomy. The surface may be based on topographical data, such as MRI, X-ray, CT or CAT scan, or ultrasound images, of the patient's anatomy. Further, the devices and systems include one or more surfaces structured to guide an orthopedic preparation device in altering or otherwise resurfacing a bone surface to receive an implant. For example, a reamer may be guided in a pre-planned insertion angle and depth relative to the surfaces of the joint, such as an acetabulum. Further disclosed herein are methods of making and using patient-matched orthopedic surgical guides.

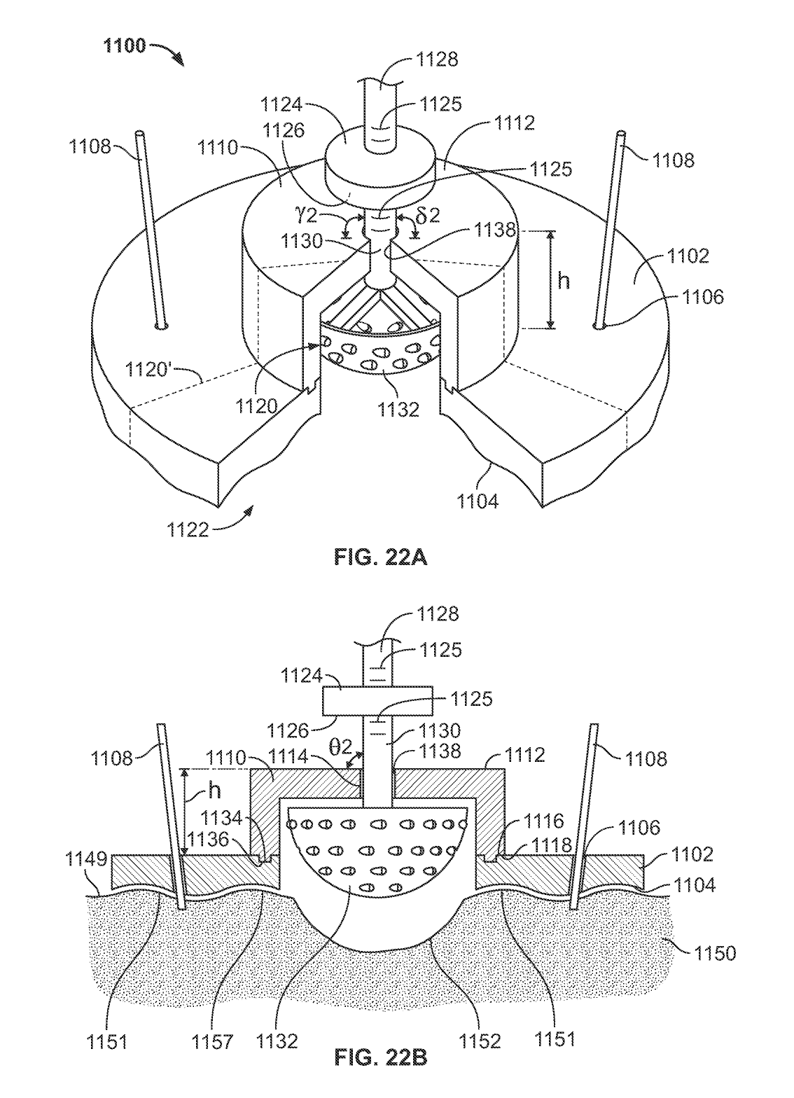

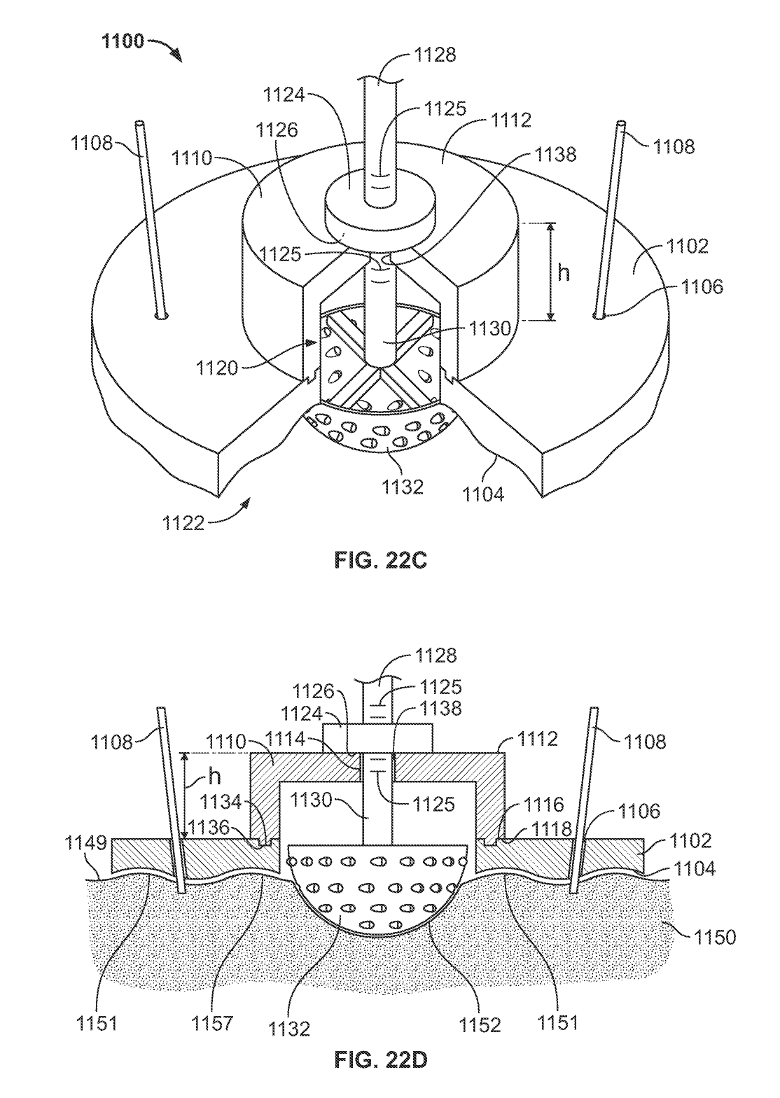

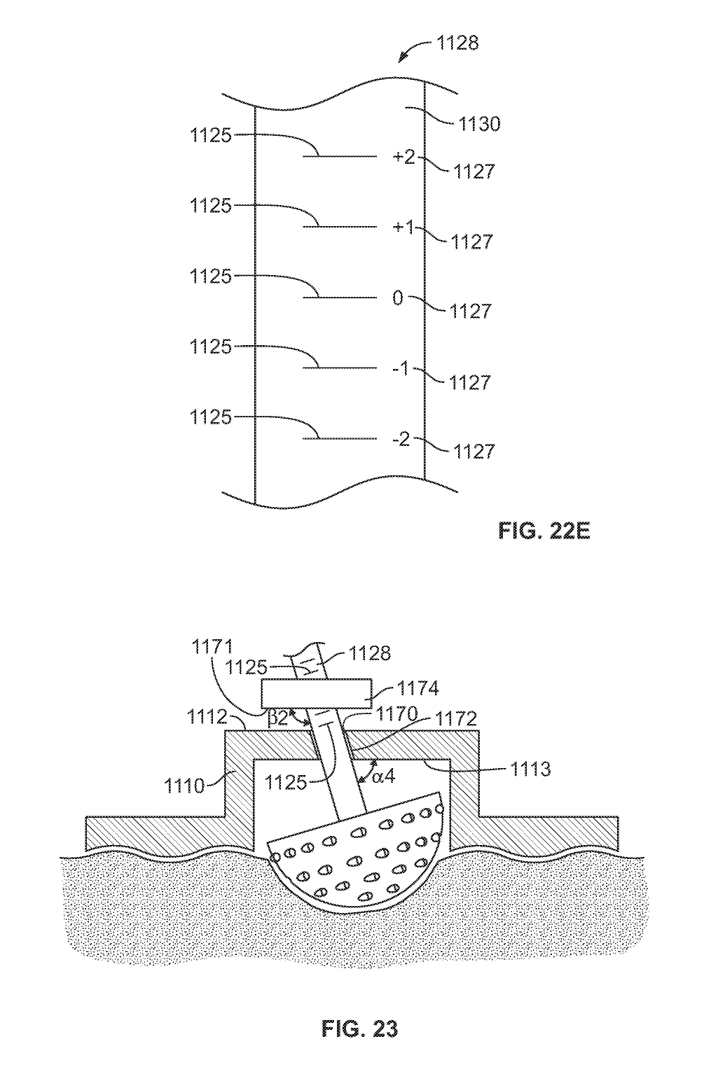

FIGS. 22A-22E show an example of an orthopedic surgical guide 1100 used to guide an orthopedic surgical preparation device, such as a reamer 1128. The guide 1100 may also be used with other orthopedic surgical preparation devices including, but not limited to, drills and impactors. The guide 1100 has a base 1102 with a lower surface 1104 structured to mate with the bone of a patient. The guide 1100 has a hub 1110 structured to receive an orthopedic surgical preparation device, such as reamer 1128. The handle or rod 1130 of the reamer 1128 is placed within the aperture 1138 of the hub 1110 and can slide along the inner guiding surface 1114. A depth stop 1124 coupled to the rod 1130 limits movement of the reamer 1128 toward the acetabulum 1152. The base 1102 of the guide 1100 typically includes one or more openings 1106 for placement of a bone pin 1108 to secure and retain the guide 1100 to the bone 1150.

In certain embodiments, the lower surface 1104 is complimentary to the bone or tissue of the patient to form a patient-matched interface. As seen, for example, in FIGS. 22B and 22D, the surface 1104 is contoured to conform to the specific anatomical features of the patient. For example, the surface forms a complementary fit 1151 with the bone surface 1149. This patient-matched fit 1151 provides a single, unique placement orientation for the guide 1000 relative to the patient. Appropriate positioning of the surface 1104 on the patient properly aligns the guide, and in particular, the inner guiding surface 1114 of the aperture 1138 within the hub 1110. The surface 1104 may have portions that fit to anatomical landmarks, portions of the acetabulum or acetabular rim, defects, or other identifiable features to rapidly and securely place the guide. The surface 1104 may extend around selected portions of rim 1157 of the acetabulum 1152, but not around the entire rim 1157. For example, in certain embodiments, the guide 1100 has lateral openings 1120 in the hub 1110 and lateral opening 1122 in the base 1102. In alternative embodiments, the surface 1104 extends around the entire rim 1157 of the acetabulum 1152. In certain implementations, the surface 1104 includes patient-matched portions that extend beyond or outside the rim 1157 of the acetabulum 1152 to form a complementary fit with portions other than acetabulum 1152 or the rim 1157. The surface 1104 may be formed based on topographical data indicative of the patient's anatomy. For example, an MRI, X-ray, CAT scan, or ultrasound images may be used to form a model of the patient's anatomy. In certain embodiments, the model is a physical model. Alternatively, the model may be a computer model.

The hub 1110 of the guide 1100 receives and guides the reamer 1128. As seen in FIGS. 22A through 22D, the surface 1114 of the aperture 1138 couples with the rod 1130 to provide a guiding interface. The reamer 1128 can be slid up or down along the inner guiding surface 1114 within the aperture 1138 of the hub 1110. In the embodiment of FIGS. 22A-22D, the aperture 1138 directs the reamer in a specific insertion angle θ2. The angle θ2 is an insertion angle within the 3-dimensional space surrounding and including the guide 1100 and the acetabulum 1152. The angle θ2 can be defined by a plurality of angles between the rod 1130 and the guide 1100 or acetabulum 1152, such as angles γ2 and angle δ2, which are located in orthogonal planes (i.e., 90° apart). A depth stop 1124 is fixedly coupled to the rod 1130 of the reamer 1128. In certain embodiments, the depth stop 1124 is removable or adjustable. The depth stop 1124 acts as a positive depth stop by physically limiting the reaming depth to prevent excessive reaming. As the reamer 1128 slides through the aperture 1138 toward the acetabulum 1152, the bottom surface 1126 of the depth stop 1124 will eventually abut the upper surface 1112 of the hub 1110, thereby preventing further reaming. For example, as shown in FIGS. 22A and 22B, the reamer 1128 begins in a raised position, wherein the reaming surface 1132 is away from the bone or tissue. As the reamer 1128 is lowered toward the acetabulum 1152, the reaming surface 1132 reams the acetabulum 1152 to form a concave, semispherical, smooth surface. Reaming can proceed until, as shown in FIGS. 22C and 22D, the reamer is in a lowered position where the depth stop 1124 abuts the upper surface 1112 of the hub 1110 to prevent further reaming.

In certain embodiments the hub 1110 is releasably attachable to the base 1102. For example, the lower surface 1116 of the hub 1110 can be mated with the upper coupling surface 1118 of the base 1102. In certain embodiments, the hub 1110 and base 1102 are coupled with a tongue and groove mechanism. For example, the hub 1110 has a tongue 1134 that fits in a groove 1136 of the base 1102. Alternatively, the hub may have a groove and the base may have a tongue. In other embodiments, alternative coupling mechanisms for the hub 1110 and the base 1102 are used, as further discussed below. The system may be provided with a plurality of hubs. For example, hubs of different height “h” may be provided to allow the surgeon to adjust the reaming depth. In alternative embodiments, a unique hub may be provided for different parts of the procedure, such as reaming, drilling, impacting, or cutting. In certain embodiments, a set of standard hubs with predefined insertion angles and heights may be provided.

As seen in FIGS. 22A and 22C, the hub 1110 includes a lateral opening 1120. The base 1102 also includes a lateral opening 1122. The hub lateral opening 1120 and the base lateral opening 1122 allow the reamer 1128 to be placed within the aperture 1138. The base 1102 and the hub 1110 may be constructed of any suitable material including, but not limited to, plastics, polymers, metals, and ceramics. In certain embodiments, the hub 1110 is constructed of a flexible material such that the hub 1110 can be flexed to expand the lateral opening 1120 to position the rod 1130 of the reamer 1128 within the aperture 1138. In other embodiments, the hub 1110 is constructed of an inflexible material and the guide 1100 is slid over the top of the reamer 1128. Alternatively, the lateral openings 1120 and 1122 may be wider than the rod 1130 and reaming surface 1132 of reamer 1128 so that the reamer 1128 can be positioned within the aperture 1138. In certain embodiments, the lateral openings 1120 and 1122 are sufficiently large such that the reaming surface 1132 can pass through the openings to exchange or remove reamer 1128 without removing the hub 1110. For example, the lateral openings 1120 and 1122 may extend to wide edge 1120′, which is wider than the reaming surface 1132 of the reamer 1128.

The lateral openings 1120 and 1122 provide a viewing window into the surgical site. The surgeon can see the reaming surface 1132 and thereby view the reaming progress to ensure the procedure is proceeding as planned and, if necessary, make intra-operative adjustments. For example, a surgeon may choose to use a different hub to adjust a reaming angle or depth.

The position, angle, and depth of reaming are typically determined in coordination with a surgeon in a pre-operative plan before the procedure starts. In certain embodiments, the pre-operative plan defines the desired reaming depth. The reaming depth may be determined from patient data, such as an MRI, X-ray, CAT scan, or ultrasound. The reaming depth, in turn, determines placement of the depth stop 1124, which is fixedly coupled to the rod 1130 of the reamer. The depth stop 1124 may be adjustably positionable along the rod 1130, for example, to provide adaptation during a procedure, while prohibiting excessive reaming. The desired reaming depth may be used to determine the height “h” of the hub 1110. The placement of depth stop 1124 and height “h” define the final depth position of the reaming surface 1132 relative to the acetabulum 1152.

The reamer 1128 may also be provided with indicators 1125 along rod 1130. In certain embodiments, the indicators 1125 correspond to different surgical options for placement of the depth stop 1124 and, in turn, the depth of reaming. As shown in FIG. 22E, the indicators 1125 can include markings 1127 corresponding to different surgical options. The markings may be alphanumeric (A, B, C, or −1, −2, 0, +1, +2, etc.), may represent different depths, and may be spaced in graduated increments, such as 2 millimeters. For example, the “0” mark may correspond to the default implant prescribed during the pre-operative planning stage. However, the surgeon may decide that adequate coverage will not be achieved with the “0” mark, and may decide to ream to the “+1” mark. In this case, the surgeon, technician, or other user can adjust depth stop 1124 to the “+1” setting. The pre-operative plan may include the use of different implants (e.g., larger or smaller cup size) for use at the different reaming depth options. For example, the surgeon may use an implant with a larger cup when reaming is done at the “+1” level. In an alternative scenario, the surgeon may have concern about weakening of the acetabular walls, and decide to ream only to the “−1” level. In this case, the pre-operative plan may include an alternative implant with a smaller cup size or an augment for use with the “−1” setting.

The pre-operative plan may define desired abduction and anteversion angles for the cup placement, which, in turn, determines the insertion angle θ2 at which the reamer will be inserted. The insertion angle θ2 may be any angle for appropriate positioning of an orthopedic surgical preparation device. For example, insertion angle θ2 may correspond to abduction and anteversion angles for a specific patient. The abduction and anteversion angles may be determined from patient data, such as an MRI, X-ray, CAT scan, or ultrasound. In certain embodiments, abduction and anteversion angles are based on predetermined target angles, such as a 45° abduction angle and a 15° anteversion angle. As shown in FIGS. 22A-22D, the inner guiding surface 1114 couples with the rod 1130 of the reamer 1128 to define the angle θ2 at which the reamer enters the acetabulum. The guiding surfaces 1114 may be structured at any appropriate angle θ2 as required for the desired abduction and anteversion angles. The angle θ2 is an angle in 3-dimensions and can be defined by a plurality of angles between the rod 130 and the guide 100 or acetabulum 152, such as angle γ2 and angle δ2, which are located in orthogonal planes (i.e., 90° apart).

FIG. 23 depicts an example of an embodiment with an alternative guiding angle for a reamer or other orthopedic device. Aperture 1170 has an angled surface 1172 which interfaces with the rod 1130 of the reamer 1128 to insert the reamer 1128 at an angle α4 relative to the hub 1110. In this example, the depth stop 1174 may also be angled at angle β2 with respect to the reamer, as desired, so that as the reamer 1128 progresses, the bottom surface 1171 of depth stop 1174 forms a flush interface with the hub 1110. In the depicted case, the upper surface 1112 of the hub 1110 is generally flat and substantially parallel to the lower surface 1113 of the hub 1110 so the angle β2 is approximately the same as angle α4. In alternative embodiments, the angle β2 is substantially different from angle α4. In certain embodiments, the upper surface 1112 and the lower surface 1113 are substantially nonparallel.

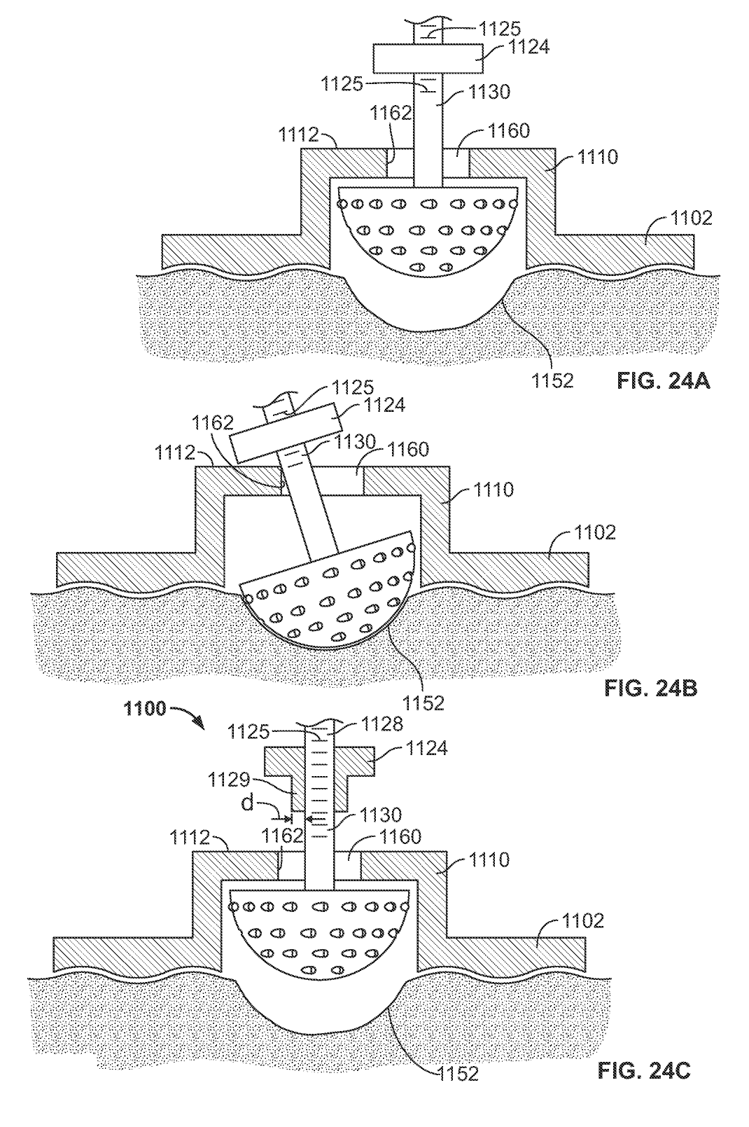

In other embodiments, a wider aperture may be used to allow freedom to adjust an insertion angle. For example, as shown in FIGS. 24A and 24B, the aperture 1160 is wider than the diameter of the rod 1130. The wide aperture 1160 allows the surgeon to ream the bone surface in a plurality of angles. The surgeon may tilt the reamer 1128 as seen in FIG. 24B. The reamer can be tilted until the rod 1130 abuts the inner guiding surface 1162 of the aperture 1160 or the stop 1124 abuts the upper guiding surface 1112. In certain embodiments, the dimensions of aperture 1160 provide a target range for the insertion angle, such as a 30-50° abduction angle and a 5-25° anteversion angle, but allow the surgeon to intra-operatively determine the most appropriate reaming and cup placement angle.

FIG. 24C depicts the guide 1100 with a sleeve 1129 that extends along the rod 1130 of the reamer 1128 and effectively increases the diameter of the rod 1130 to adjust and limit the range of possible reaming angles. The sleeve 1129 may be provided separately from the depth stop 1124, or in certain embodiments, is integrally coupled with depth stop 1124. The sleeve 1129 may be adjustably positionable along the rod 1130. The sleeve 1129 may be aligned with indicators 1125. The sleeve 1129 may also be removable and interchangeable with sleeves having different sizes and diameters (“d”). As the reamer 1128 is inserted into the acetabulum 1152, the sleeve 1129 enters the aperture 1160 and limits the range of possible reaming angles during reaming by decreasing the space between the reamer 1128 and the surface 1162 of aperture 1160. With a larger diameter “d” of the sleeve 1129, the range of reaming angles decreases. In certain embodiments, the surgeon is provided with a plurality of sleeves on a surgical tray, with each sleeve corresponding to a different level of adjustability during reaming. For example, a first sleeve may provide a 5° range of reaming angles and a second sleeve may provide a 10° range of reaming angles. Additional sleeves corresponding to alternative ranges of angles may also be provided at any appropriate increment or range. The surgeon may choose an appropriate sleeve for the surgical procedure and may use different sleeves at different times during the surgical procedure. In certain embodiments sleeve 1129 is provided with indicia markings. In alternative embodiments, the sleeve 1129 may be tapered.

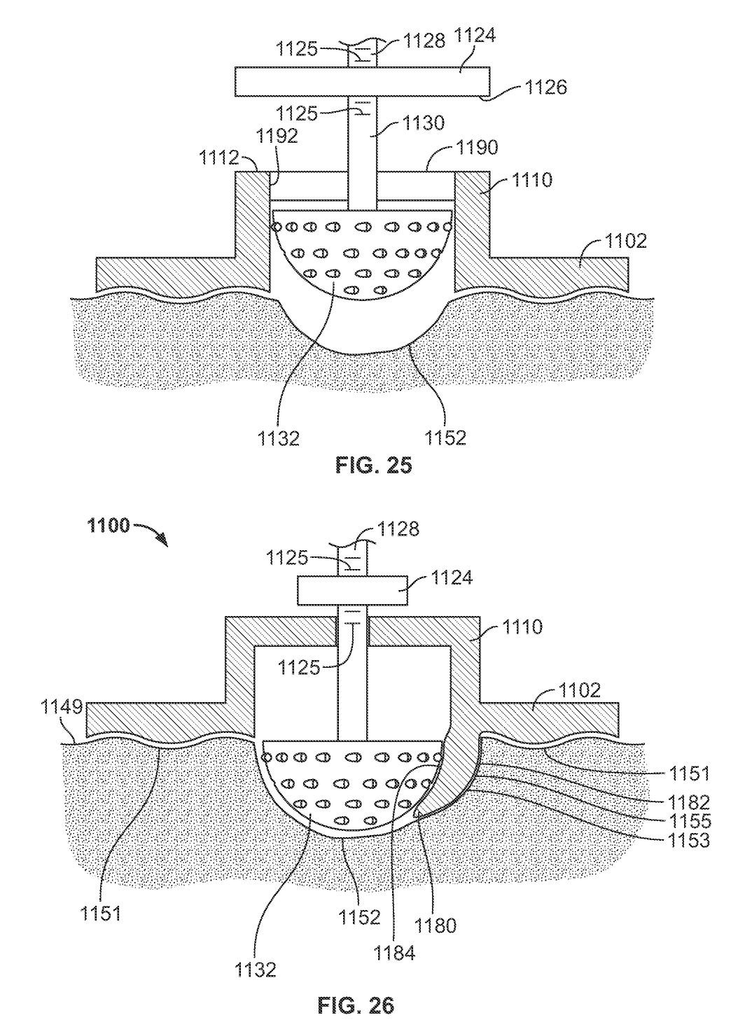

In alternative embodiments, the aperture of the hub guides the cutting portion of the reamer instead of the rod of the reamer. For example, as seen in FIG. 25, the hub 1110 has a wide aperture 1190 with an inner guiding surface 1192 that guides the reaming surface 1132 of the reamer 1128. The inner guiding surface 1192 of the aperture 1190 may also be formed in various angles for guiding the reamer to the appropriate reaming position. In use, the reaming proceeds as the reamer 1128 is lowered through the aperture 1190 towards the acetabulum until the bottom surface 1126 of the depth stop 1124 abuts the upper surface 1112 of the hub 1110. That abutment limits the depth of the reaming within the acetabulum 1152.

FIG. 26 shows an example of a guide 1100 with an extension 1180 of the base 1102 that extends into the acetabulum 1152 to help protect weakened or vulnerable areas 1153 within the acetabulum 1152. For example, portion 1153 may be a defect, such as a deformity, weak area, or significantly deteriorated area of the acetabulum 1152. Paprosky et al. describes acetabular defects, and is hereby incorporated by reference herein (W.G. Paprosky, et al. Journal of Arthroplasty, vol. 9, 1994, pp 33-44). Alternatively, portion 1153 may contain vasculature that, if disturbed, could cause bleeding. The extension 1180 has a coupling surface 1182 that mates with a wall or protected portion 1153 of the acetabulum 1152 and forms a complementary fit 1155, in addition to the complementary fit 1151 along the bone surface 1149 formed with surface 1104. The extension 1180 thereby provides additional means of securely coupling the guide 1100 with the acetabulum. In certain embodiments, the extension 1180 is the primary means of securely placing the guide 1100 and provides a single, unique placement orientation for the guide 1100 relative to the acetabulum 1152. For example, the extension coupling surface 1182 of the extension 1180 may be the only patient-matched surface of the guide 1100. The extension 1180 also has an outer surface 1184 that prohibits the reaming surface 1132 of the reamer 1128 from cutting the protected portion 1153 of the acetabulum 1152.

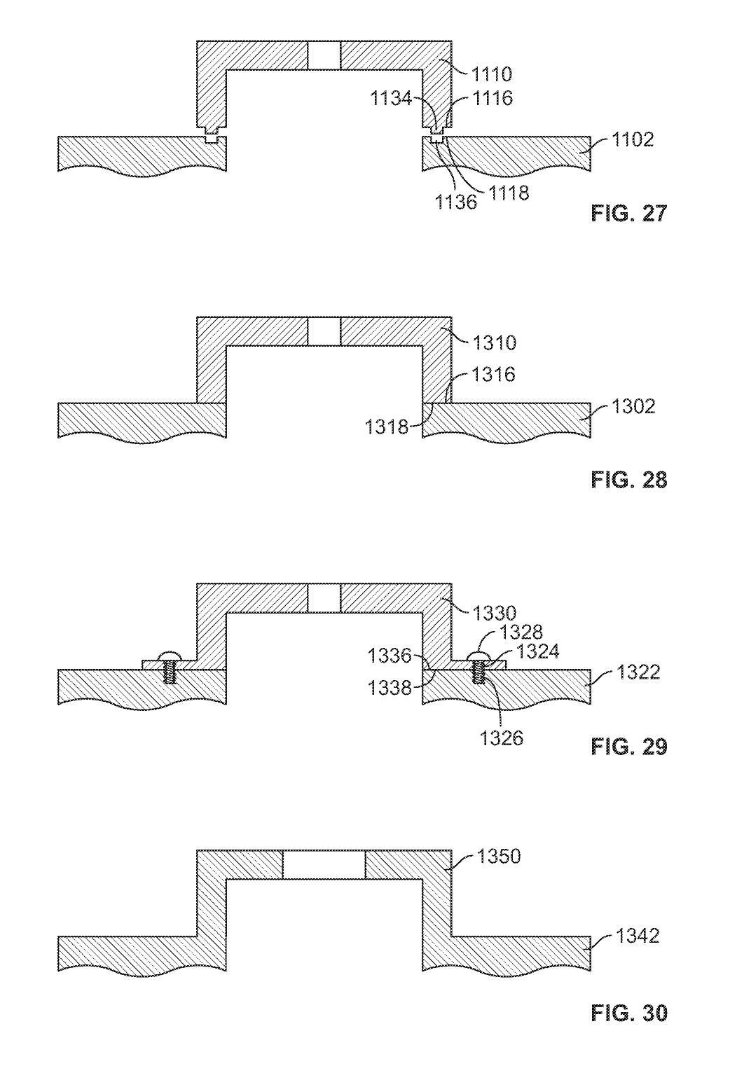

FIGS. 27 through 30 show various mechanisms to couple the hub of the guide with the base of the guide. For example, as seen in FIG. 27, the hub 1110 has a lower surface 1116 with a tongue 1134 that interfaces with the groove 1136 of the coupling surface 1118 of the base 1102. In other embodiments, the hub may have a groove and the base may have a tongue to fit into the groove. When the tongue is placed within the groove, the hub 1110 is fixed in place with the base 1102. In certain embodiments, the hub 1110 is removable from the base 1102. The base 1102 may be compatible with alternative hubs, such as a set of standard hubs having predetermined dimensions for guiding a surgical preparation device.

FIG. 28 shows an example of a hub 1310 with a lower surface 1316 coupled to an upper surface 1318 of a base 1302. After aligning the hub 1310 and base 1302, the hub 1310 and the base 1302 are coupled by a glue, epoxy, cement, weld, adhesive, or other fastening mechanism.

FIG. 29 shows an example of a hub 1330 coupled to a base 1322 by means of a screw 1328. In this embodiment, the hub 1330 has an opening 1324 that is aligned with an opening 1326 in the base 1322. A screw 1328 is inserted in the openings within the hub 1330 and the base 1322 in order to securely couple the hub 1330 and base 1322 together. The screw may be released to decouple the hub 1330 from the base 1322. As indicated with other embodiments, the base 1302 may be compatible with alternative hubs, such as a set of standard hubs having predetermined dimensions for guiding a surgical preparation device. Other fastening means, including pins, holes, snaps, flanges, friction fits, and clamps may also be used.

A fixedly coupled hub 1350 and base 1342 may be formed according to a pre-operative plan for a specific patient, for example, as shown in FIG. 30. In certain embodiments, the hub 1350 and the base 1342 are formed together as a single, unitary piece. For example, the hub 1350 and base 1342 may be formed from rapid-prototyping methods, molds, or other manufacturing techniques.

FIGS. 31-34 show embodiments of depth stops that limit the reaming depth at the surgical site. For example, FIG. 31 shows a depth stop 1124 which is a sleeve around the rod 1130 of the reamer. The bottom surface 1126 of the depth stop 1124 abuts the upper surface 1112 of the hub 1110 to limit the movement of the reamer 1128. In certain embodiments the sleeve or depth stop 1124 is adjustably positionable along the rod 1130, for example by a friction fit, latch, or other connection means within or along the rod 1130. The depth stop 1124 may be moved up or down along the rod 1130 of the reamer 1128. The depth stop 1124 may be constructed of any suitable material including, but not limited to, plastics, polymers, metals and ceramics. The placement of the sleeve or depth stop 1124 is determined in the pre-surgical planning In other embodiments, the depth stop 1124 is fixedly attached to the rod 1130 of the reamer 1128.

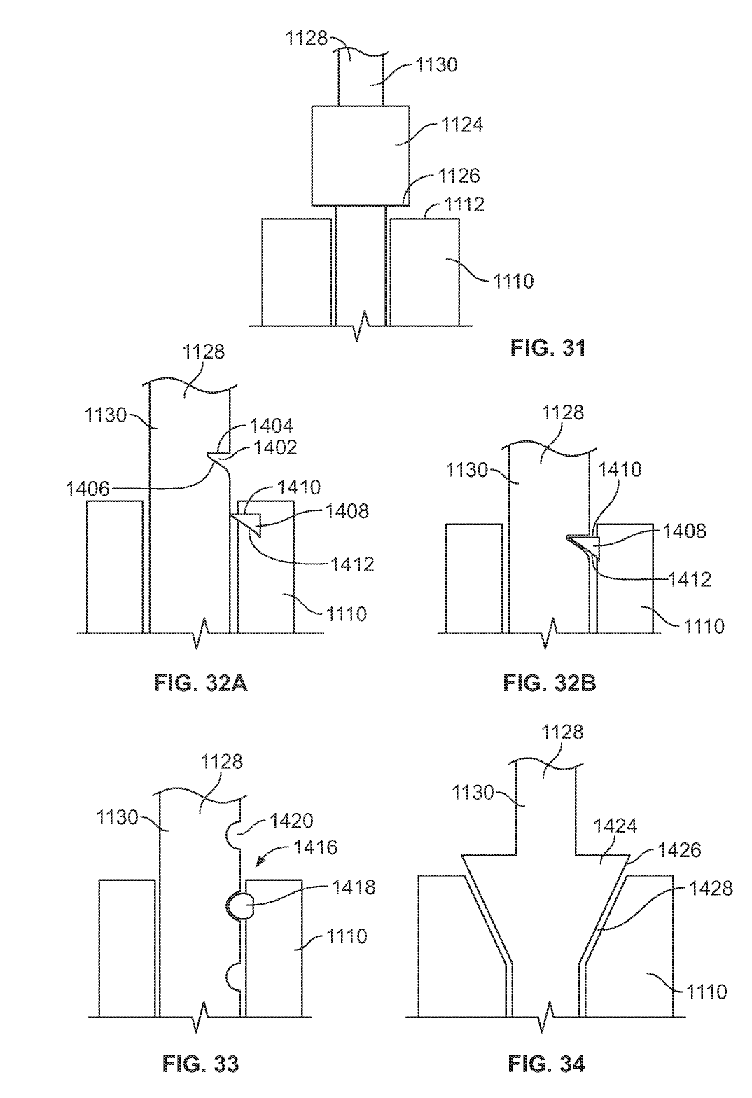

In certain embodiments, the depth stop is included with the hub. As shown in FIGS. 32A-32B, the depth stop is a latch mechanism with a stop component on the hub 1110. More particularly, the rod 1130 has a notch 1402 and the hub 1110 has a latch 1408. As the rod 1130 is slid along the aperture 1138 of the hub 1110, the latch 1408 engages the notch 1402 of the rod 1130, as seen in FIG. 32B. In certain embodiments, the latch 1408 is spring-loaded to protrude into the notch 1402 when the latch 1408 and notch 1402 are aligned. When the latch 1408 engages the notch 1402, the upper surface 1404 of the notch 1402 abuts upper surface 1410 of the latch 1408 to prohibit the reamer 1128 from moving further down the aperture into the acetabular site. In certain embodiments, the latch 1408 is releasable. For example, if the rod 1130 is moved upward, the lower surface 1406 of the notch pushes lower surface 1412 of the latch 1408 upwardly to cause latch 1408 to recede laterally into the hub 1110.

FIG. 33 shows a ball latch mechanism 1416. As the rod 1130 is slid along the aperture 1138, the ball 1418 in the hub 1110 engages a detent surface 1420 within the rod 1130. In certain embodiments, the ball 1418 is spring-loaded to protrude into the receiving surface 1420 when the latch 1418 and receiving surface 1420 are aligned. The latch mechanism 1416 may be releasable. If sufficient force is applied to the rod 1130 either direction, the detent surface 1420 will push the ball 1418 into the hub 1110. The depth stop serves as a safety mechanism, depth indicator, or warning, which still allows the surgeon to continue. In certain embodiments, the rod 1130 has a plurality of detent surfaces 1420 which provide the surgeon with tactile feedback of the reaming depth. The plurality of detent surfaces 1420 also provides the surgeon with flexibility to determine the appropriate reaming depth during the procedure.

The depth stops may take other forms. For example, FIG. 34 shows an example of a reaming rod 1130 configured with a distal wedge depth stop. The depth stop includes an angled surface 1426 on a wedge 1424, which couples with an angled surface 1428 of the hub 1110. In certain embodiments, the wedge 1424 has the shape of a cone, and the angled surface 1428 of the hub 1110 is complementary to that surface. The wedge 1424 may be adjustably positionable along the rod 1130 of the reamer 1128, for example by a friction fit, latch, or other connection means within or along the rod 1130. In other embodiments the wedge is fixedly attached to the rod 1130.

Combinations of depth stops may also be used. For example, a releasable latch depth stop, such as system 1416 shown in FIG. 33, may be used to provide a depth indicator to the surgeon and combined with an absolute depth stop, such as those shown in FIGS. 31 and 34, further up the rod 1130 to ensure an absolute reaming depth is not exceeded.

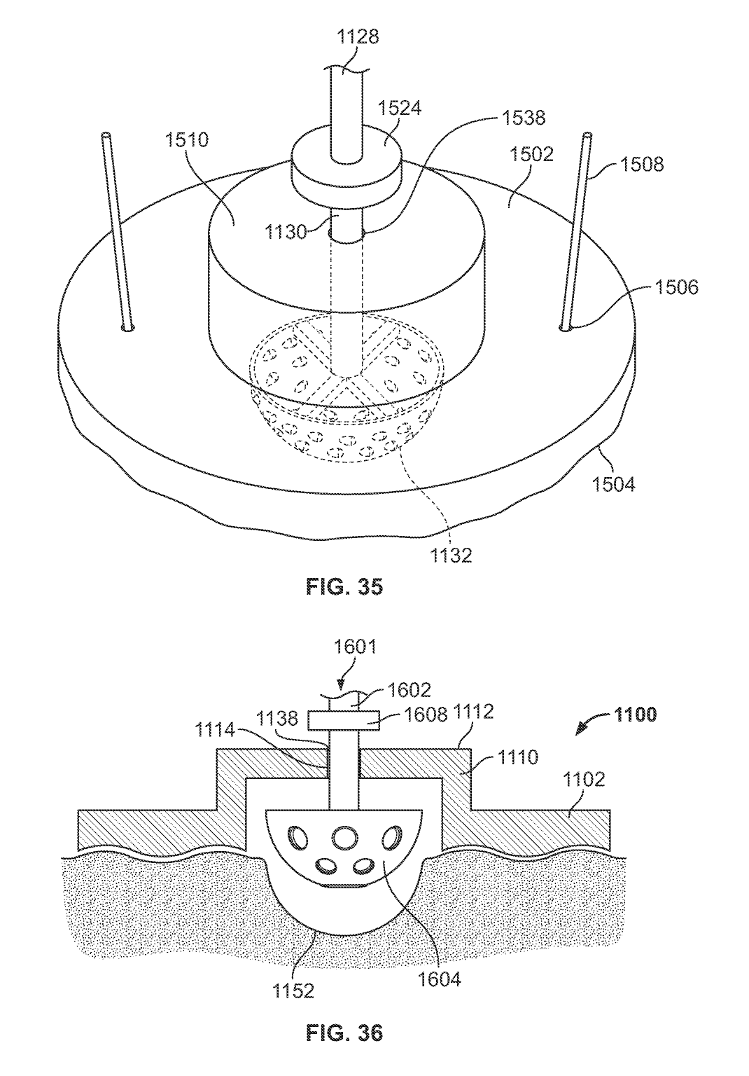

FIG. 35 shows an example of a surgical guide 1500 which fully encloses an orthopedic surgical preparation device such as reamer 1128. The guide 1500 is similar to other embodiments discussed, in that it includes a base 1502, a hub 1510 and a depth stop 1524. The base also includes a patient interface surface 1504 structured to form a complimentary fit with a specific portion of the patient's anatomy. The base may also include an opening 1506 for receiving and retaining a bone pin 1508 which fastens the base 1502 to the patient's bone or tissue. Base 1500 does not include a lateral opening in the hub or the base as seen in previous embodiments. The guide 1500 is typically slid over the reamer 1128 such that the rod 1130 of the reamer 1128 is positioned within the aperture 1538 of the guide 1500.

Although the systems and methods described herein have primarily been described for use with a reamer, they may also be used with other surgical preparation devices, such as drills and impactors. For example, as seen in FIG. 36, guide 1100 can be used with an impactor 1601. Impactor 1601 has an impactor rod 1602 that fits within the aperture 1138 and is guided by the inner guiding surface 1114 of the aperture 1138. The impactor 1601 may be used to place a shell implant 1604 in the acetabulum 1152. As shown in other embodiments, a depth stop 1608 may be used to limit the impaction depth as the depth stop 1608 interfaces with the upper surface 1112 of the hub 1110. Similarly, a drill could be used with the guide system to accurately drill in a specified position to a specified depth.

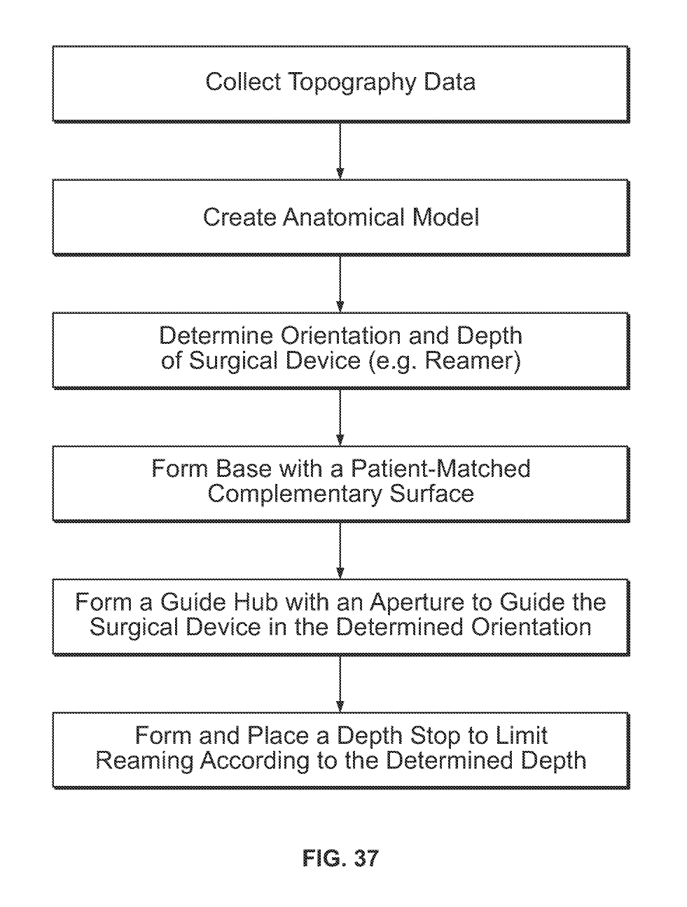

FIG. 37 describes a method of making an orthopedic surgical guide for a specific patient. Initially, topography data of a portion of a patient's anatomy is taken, for example by imaging techniques including, but not limited to, MRI, X-ray, CAT scan, and ultrasound. The data is then used to create a model of the patient's anatomy from the data. In certain embodiments, the model is a physical model or replica of the portion of the patient's anatomy. In alternative embodiments, the model is a computer model, such as a 3-dimensional image. The model is used to develop a pre-operative plan and determine the orientation and depth of the surgical preparation device, such as a reamer. Preferably, the plan defines abduction and anteversion angles for the placement of a prosthetic acetabular cup, which, in turn, determines the orientation of the reamer. The base is formed with a patient-matched surface that forms a complementary fit with the patient's anatomy. The guide hub is formed using the pre-operative plan with an aperture structured and positioned to guide the surgical device in the determined orientation. Additionally, a depth stop is formed, such as those previously discussed, and positioned to limit reaming according to the determined depth in the pre-operative plan.

The systems, devices, and methods of the present disclosure allow for guiding surgical instruments to prepare a bone surface for joint reconstruction or repair. As discussed above, example devices and systems include a surgical guide system having a base and at least one linearly repositionable leg that provides tactile depth feedback and a limiting depth stop for an orthopedic preparation device, such as a reamer. Further example devices and systems include a surgical guide that operates with a positive depth stop for an orthopedic reaming or impacting device. The disclosed features of the present disclosure may be implemented, in any combination and subcombination (including multiple dependent combinations and subcombinations), with one or more other features described herein. For example, in certain embodiments, features of the examples above are combined into a single surgical guide system, whereby the surgical guide system includes at least one linearly repositionable leg that provides tactile depth feedback and a limiting depth stop and which also includes a positive depth stop for an orthopedic reaming or impacting device.

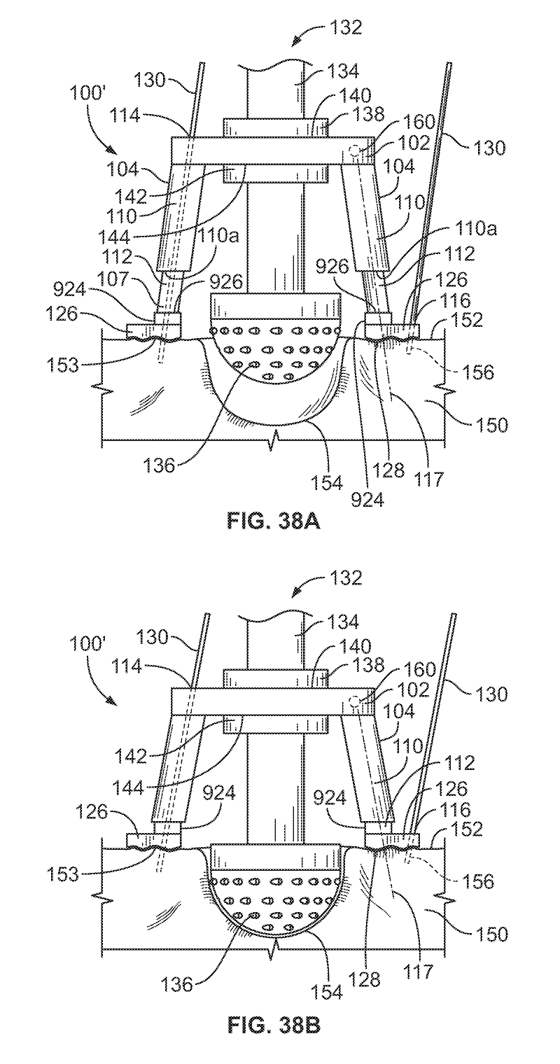

FIGS. 38A and 38B show side elevation views of a surgical guide 100′ with moveable legs for guiding a surgical preparation device and depth stops, according to certain embodiments. These figures depict the guide 100 described above in connection with FIGS. 2B and 2C, but the device 100′ further includes depth stops 924 at the distal end 107 of the respective legs 104. In FIG. 38A, the reamer 132 is in a raised position. The hub 102 is repositionable relative to the distal end 107 of the leg 104 through the movement of the upper portion 110 and lower portion 112 of the leg. When a sufficient force is applied along the reamer 132 downwards toward the acetabulum 154, the upper portion 110 of the leg 104 moves relative to the lower portion 112 of the leg 104 along axis 117 to move the cutting portion 136 of the reamer 132 into contact with the acetabulum 154. The depth of the reaming is controlled by operational range of motion between upper portion 110 and lower portion 112, which provides a positive depth stop for the reaming process at the limit of the allowed motion. As shown in FIGS. 38A and 38B, the depth of the reaming is also controlled by the depth stops 924.

The depth stops 924 coupled to the distal end 107 of the respective legs 104, near the foot 126 of the legs 104, limits movement of the reamer 132 toward the acetabulum 154. The depth stops 924 are fixedly coupled to the lower portion 112 of the legs 104. In certain embodiments, the depth stops 924 are removable or adjustable. In certain embodiments, one depth stop may be used rather than the two shown, or in some cases more than two depth stops can be used if there are additional legs. The depth stops 924 act as a positive depth stop by physically limiting the reaming depth to prevent excessive reaming. As a force is applied along the reamer 132 towards the acetabulum 154, the top surface 926 of the depth stops 924 will eventually abut the bottom surface 110a of the upper portion 110 of the leg 104, thereby preventing further reaming. For example, as shown in FIGS. 38A and 38B, the reamer 132 begins in a raised position, wherein the cutting portion 136 is away from the bone or tissue. As the reamer 132 is lowered toward the acetabulum 154, the cutting surface 136 reams the acetabulum 154 to form a concave, semispherical, smooth surface. Reaming can proceed until, as shown in FIG. 38B, the reamer 132 is in a lowered position where the depth stops 924 abut the lower surface 110a of the upper portion 110 of the legs 104 to prevent further reaming. The depth stops 924 can therefore control depth of reaming in addition to the resistive forces discussed above in connection with operation of the legs 104.

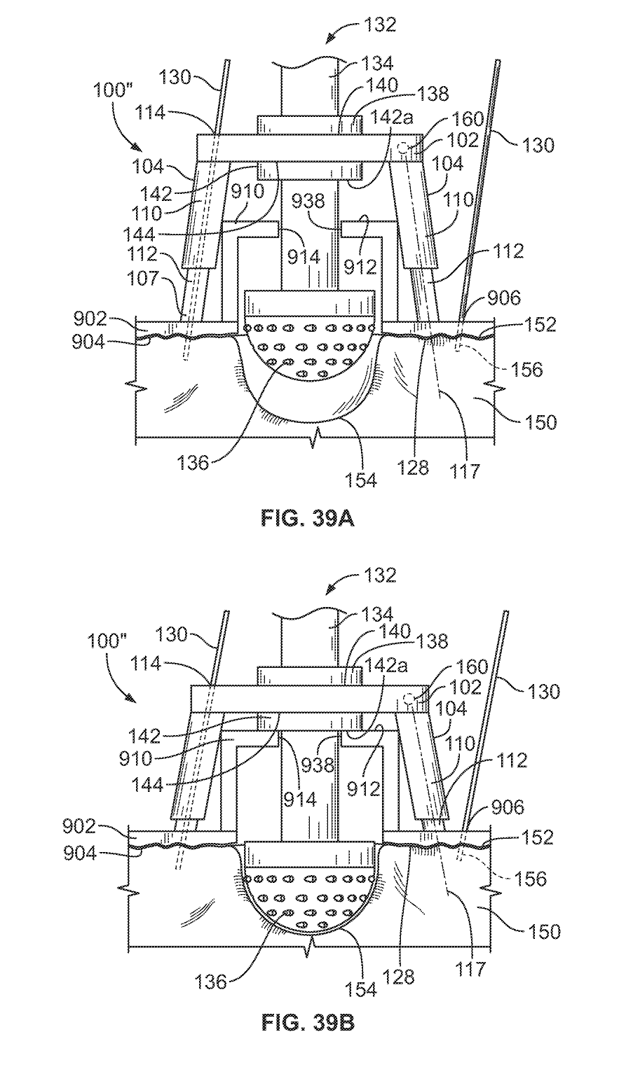

FIGS. 39A and 39B show side elevation views of a surgical guide 100″ with moveable legs for guiding a surgical preparation device and a depth stop for use with an orthopedic surgical preparation device, such as a reamer, according to certain embodiments.

These figures depict the guide 100 described above in connection with FIGS. 2B and 2C, but the device 100″ further includes a base 902 with a lower surface 904 structured to mate with the bone of a patient. The guide 100″ also has a hub 910 structured to receive an orthopedic surgical preparation device, such as reamer 132. The handle or rod 134 of the reamer 132 is placed within the aperture 938 of the hub 910 and can slide along the inner guiding surface 914. The retaining sleeves 138 and 142, which are tightly coupled to the rod 134 of the reamer 132, can thereby serve as a depth stop coupled to the rod 134 that limits movement of the reamer 132 toward the acetabulum 154. The base 902 of the guide 100″ typically includes one or more openings 906 for placement of a bone pin 130 to secure and retain the guide 100″ to the bone 150.

The hub 910 of the guide 100″ receives and guides the reamer 132. As seen in FIGS. 39A and 39B, the surface 914 of the aperture 938 couples with the rod 134 to provide a guiding interface. The reamer 132 can be slid up or down along the inner guiding surface 914 within the aperture 938 of the hub 910. A depth stop may be fixedly coupled to the rod 134 of the reamer 132. Here, the retaining sleeves 138 and 142 can act as a positive depth stop. In certain embodiments, retaining sleeves 138 and 142 are removable or adjustable. The sleeves 138, 142 act as a positive depth stop by physically limiting the reaming depth to prevent excessive reaming. As the reamer 132 slides through the aperture 938 toward the acetabulum 154, the bottom surface 142a of the sleeve 142 will eventually abut the upper surface 912 of the hub 910, thereby preventing further reaming. For example, as shown in

FIG. 39A, the reamer 132 begins in a raised position, wherein the cutting surface 136 is away from the bone or tissue. As the reamer 132 is lowered toward the acetabulum 154, the cutting surface 136 reams the acetabulum 154 to form a concave, semispherical, smooth surface. Reaming can proceed until, as shown in FIG. 39B, the reamer is in a lowered position where the depth stop (sleeve 142) abuts the upper surface 912 of the hub 910 to prevent further reaming. The hub 910 and sleeves, acting as depth stops, can therefore control depth of reaming in addition to the resistive forces discussed above in connection with operation of the legs 104.

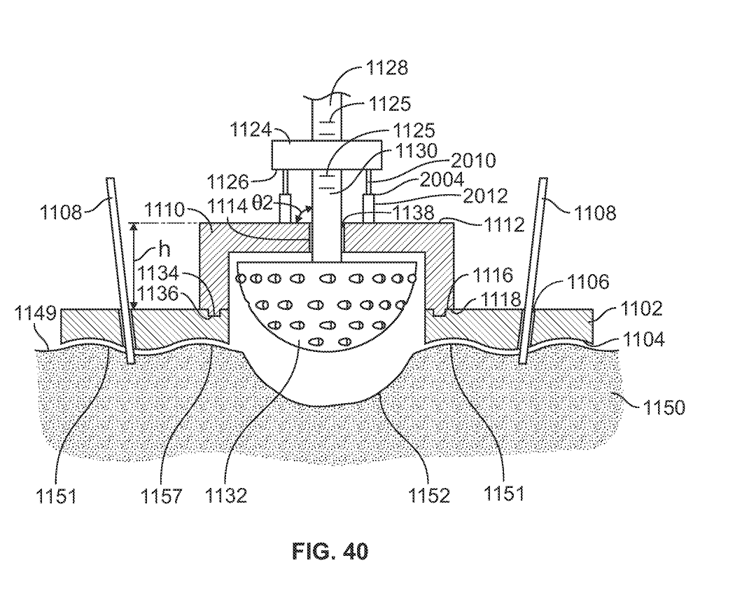

FIG. 40 shows a cross-sectional view of a surgical guide with moveable legs for guiding a surgical preparation device and a depth stop for use with an orthopedic surgical preparation device, such as a reamer, according to certain embodiments. This figure depicts the guide described above in connection with FIG. 22B, but the device further includes legs 2004 between the depth stop 1124 and the hub 1110. In certain embodiments, the legs 2004 are fixedly coupled to the depth stop 1124 and the hub 1110. The legs 2004 may also be removable from the depth stop 1124 and/or hub 1110. The legs 2004 comprise an upper portion 2010 and a lower portion 2012, which are linearly repositionable relative to each other along their common longitudinal axis. In certain embodiments, as shown in FIG. 40, the guide includes a plurality of legs 2004.

The upper portion 2010 of the leg and lower portion 2012 of the leg 2004 are repositionable relative to each other to allow the depth stop 1124 to move up and down for repositioning an orthopedic preparation device at a surgical site. FIG. 40 shows the legs 2004 in a decompressed position. The upper portion 2010 is coupled to the depth stop 1124 and the lower portion 2012 is coupled to the hub 1110 so that movement of the leg 2004 vertically repositions depth stop 1124 with respect to the patient's anatomy. As the depth stop 1124 is lowered with movement of the reamer, the leg remains fixed, and the depth stop 1124 is brought closer to the hub 1110. The movement of upper portion 2010 relative to lower portion 2012 has a limited operational range of motion. In certain embodiments the movement mechanism of the leg 2004 includes a spring or slot to control motion, provide tactile feedback, and limit the reaming depth. In certain embodiments, the angle of attachment of the legs 2004 relative to the hub 1110 is adjustable, for example, by a latch, ratchet, or other mechanical connection. In alternative embodiments, the legs 2004 are in a fixed orientation relative to the hub 1110.

When a sufficient force is applied along the reamer 1128 downwards toward the acetabulum 1152, the upper portion 2010 of the leg 2004 moves relative to the lower portion 2012 of the leg 2004 along a common axis to move the reaming surface 1132 of the reamer 1128 into contact with the acetabulum 1152. As the reaming surface 1132 progresses deeper within acetabulum 1152, the legs 2004 preferably provide increased resistance to the downward motion of the reamer 1128, for example, by compressing a spring. This resistive force provides the surgeon with tactile feedback relative to the depth of the reamer 1128 within the acetabulum 1152. The depth of the reaming is controlled by operational range of motion between upper portion 2010 and lower portion 2012, which provides a positive depth stop for the reaming process at the limit of the allowed motion. The legs 2004 can therefore control depth of reaming in addition to the positive depth stop 1124.

The foregoing is merely illustrative of the principles of the disclosure, and the systems, devices, and methods can be practiced by other than the described embodiments, which are presented for purposes of illustration and not of limitation. It is to be understood that the systems, devices, and methods disclosed herein, while shown for use in hip systems, may be applied to systems, devices, and methods to be used in other surgical procedures including, but not limited to hip arthroplasty, knee arthroplasty, spine arthroplasty, cranio-maxillofacial surgical procedures, shoulder arthroplasty, as well as foot, ankle, hand, and other extremity procedures.

Variations and modifications will occur to those of skill in the art after reviewing this disclosure. The disclosed features may be implemented, in any combination and subcombination (including multiple dependent combinations and subcombinations), with one or more other features described herein. The various features described or illustrated above, including any components thereof, may be combined or integrated in other systems. Moreover, certain features may be omitted or not implemented.

Examples of changes, substitutions, and alterations are ascertainable by one skilled in the art and could be made without departing from the scope of the information disclosed herein. All references cited herein are incorporated by reference in their entirety and made part of this application.

1-19. (canceled)

20. A method of using an orthopedic surgical guide, the guide having at least one leg coupled to a hub and a base, wherein the at least one leg has a longitudinal axis and first leg component coupled to a second leg component, wherein the first leg component is linearly repositionabli, relative to the second leg component along the longitudinal axis, wherein the hub has an aperture that receives an orthopedic surgical preparation device, and wherein the base has a surface structured to mate with a specific portion of a patient's anatomy; the method comprising:
placing the base on the specific portion of the patient's anatomy;
coupling the orthopedic surgical preparation device within the aperture of the hub;
sliding the first leg component along the longitudinal axis by applying a force on the orthopedic surgical preparation device; and
contacting the orthopedic surgical preparation device with bone or tissue.

21. The method of claim 20, further comprising positioning the at least one leg relative to the hub to adjust the angle between the at least one leg and the hub.

22. The method of claim 20, wherein the surgical guide has a depth stop structured to limit the range of movement between the first leg component and the second leg component, and wherein the method further comprises sliding the first leg component relative to the second leg component.

23-25. (canceled)

26. A method of using an orthopedic surgical guide, the guide having at least one leg, wherein the leg has a proximal end coupled to an upper housing of the guide and a distal end; the method comprising:
mating a surgical preparation device with a support surface of the upper housing;
positioning the distal end of the leg at a predetermined first distance from the upper housing; and
repositioning the upper housing with respect to the distal end of the leg, while the distal end remains fixed.

27. The method of claim 26, wherein the distal end of the leg has a surface structured to mate specifically with a portion of a patient's anatomy.

28. The method of claim 26, wherein repositioning the upper housing comprises lowering the upper housing to a second distance from the distal end of the leg, such that the first distance is greater than the second distance.

29-59. (canceled)

60. A method of using an orthopedic surgical guide, comprising:
attaching a solid base to a portion of a patient's anatomy in a predetermined configuration relative to that portion;
aligning a guide hub with respect to the base, the guide hub having an aperture for guiding an orthopedic cutting or impacting device, and a solid interface configured to mate with a depth stop to limit movement of the device relative to the guide hub;
placing the orthopedic cutting or impacting device within the aperture of the guide hub; and
sliding the orthopedic cutting or impacting device until the depth stop couples with the solid interface of the hub,

61. The method of claim 60, further comprising attaching the guide hub to the base.

62. The method of claim 60, further comprising placing the depth stop on the orthopedic cutting or impacting device.

63-65. (canceled)

66. The method of claim 20, further comprising:
guiding the orthopedic surgical preparation device in a pre-planned insertion angle and depth relative to the bone or tissue to prepare the bone or tissue for placement of an orthopedic implant; and
implanting the orthopedic implant into the prepared bone or tissue.

67. The method of claim 26, further comprising:
guiding the surgical preparation device in a pre-planned insertion angle and depth relative to a bone or tissue surface to prepare the bone or tissue surface for placement of an orthopedic implant; and
implanting the orthopedic implant into the prepared bone or tissue surface.

68. The method of claim 60, further comprising:
preparing the portion of the patient's anatomy for receiving an orthopedic implant by contacting the orthopedic cutting or impacting device with said portion; and
implanting an orthopedic implant into said prepared portion.
